(19) United States
(12) Patent Application Publication
(10) Pub. No.: US 2011/0045501 A1
(43) Pub. Date: Feb. 24, 2011

(54) ST2-BASED DENGUE FEVER DIAGNOSTIC

(76) Inventors: Irene Bosch, Brookline, MA (US); Becerra Aniuska, Worcester, MA (US)

Correspondence Address: MEDLEN & CARROLL, LLP, 101 HOWARD STREET, SUITE 350, SAN FRANCISCO, CA, 94105, US

(21) Appl. No.: 12/812,004

(22) PCT Filed: Jan. 8, 2009
(86) PCT No.: PCT/US09/00085

§ 371 Date: Nov. 8, 2010

(30) Foreign Application Priority Data
Jan. 8, 2008 (US) 61/010316

(51) Int. Cl. G01N 33/574; C07K 14/00; G01N 33/53

(52) U.S. Cl. 435/7.23; 530/350; 436/501; 436/86

(57) ABSTRACT

A cytokine receptor family member (ST2), which has membrane bound as well as soluble bound forms, is elevated in patients during acute phase of dengue fever. Moreover, secondary cases of dengue fever had even more pronounced elevation of ST2 in serum in comparison to primary cases. One possible role of soluble ST2 might be to act as decoy for membrane bound ST2 signaling, therefore, promoting a proinflammatory response in secondary dengue infections. Alternatively, ST2 may act as a biomarker for endothelial damage such that ST2 may be useful in identifying severe dengue infections, specifically in dengue hemorrhagic fever.

### STATEMENT OF GOVERNMENT SUPPORT

This invention was made with government support awarded by the National Institutes of Health (grant number NIAID #U01 A145440). The government has certain rights in the invention.

### FIELD OF INVENTION

This invention relates to the detection and diagnosis of inflammatory diseases. One such inflammatory disease is believed caused by the dengue fever virus. An infection caused by the dengue fever virus may result in conditions ranging from dengue fever, to dengue hemorrhagic fever, to dengue septic shock. In one embodiment, the soluble ST2 protein is believed to act as a biomarker for dengue fever virus infection, and may be useful as a diagnostic kit.

### BACKGROUND

Dengue virus is a single-stranded RNA mosquito-borne virus that belongs to the Flaviviridae family. It infects humans and produces a disease with a broad spectrum of clinical manifestations that ranges from an acute self-limiting febrile illness (Dengue Fever, DF) to various grades of a severe disease (Dengue Hemorrhagic Fever, DHF) that could result in a life-threatening syndrome (Dengue Shock Syndrome, DSS). Chaturvedi et al., “Dengue and dengue haemorrhagic fever: implications of host genetics” FEMS Immunol Med Microbiol 47:155-166 (2006).

Dengue virus (DV) has reemerged as a major global health problem in the tropics, particularly among children Gubler, D. J. 2001. “Human arbovirus infections worldwide” Ann N Y Acad Sci 951:13-24; and Mairuhu et al., 2004. “Dengue: an arthropod-borne disease of global importance” Eur J Clin Microbiol Infect Dis 23:425-33. This mosquito-borne flavivirus, for which there is no vaccine or anti-viral treatment, causes an estimated 50 million infections annually. “Joint WHO HQ/SEAROP/WPRO meeting on DengueNet implementation in South-East Asiand the Western Pacific, Kuala Lumpur, 11-13 Dec. 2003” Wkly Epidemiol Rec 78:346-347 (2003); and Petersen et al., “Shifting epidemiology of Flaviviridae” J Travel Med 12 Suppl 1:S3-S11 (2005). Most dengue infections result in a self limited febrile illness (i.e., for example, dengue fever, DF). Less frequently, infections can cause dengue hemorrhagic fever (DHF), a potentially fatal plasma leakage syndrome.

### SUMMARY

This invention relates to the detection and diagnosis of inflammatory diseases. One such inflammatory disease is believed caused by the dengue fever virus. An infection caused by the dengue fever virus may result in conditions ranging from dengue fever, to dengue hemorraghic fever, to dengue septic shock. In one embodiment, the soluble ST2 protein is believed to act as a biomarker for dengue fever virus infection, and may be useful as a diagnostic kit.

In one embodiment, the present invention contemplates a biomarker for a dengue fever infection, wherein said biomarker comprises a protein level elevated to at least 1.5 times that of other febrile illnesses. In one embodiment, the protein comprises a soluble interleukin 1 receptor-like 1 protein. In one embodiment, the elevated protein level is detected in a biological sample. In one embodiment, the biological sample is selected from the group comprising whole blood, plasma, serum, or a tissue biopsy. In one embodiment, the tissue biopsy comprises a somatic cell. In one embodiment, the dengue fever infection comprises symptoms including but not limited to, headache, joint aches, muscle aches, nausea, swollen lymph nodes, and/or vomiting.

In one embodiment, the present invention contemplates a biomarker for a dengue fever infection, wherein said biomarker comprises a nucleic acid level elevated to at least 1.5 times that of other febrile illnesses. In one embodiment, the nucleic acid encodes a soluble interleukin 1 receptor-like 1 protein. In one embodiment, the elevated nucleic acid level is detected in a biological sample. In one embodiment, the biological sample is selected from the group comprising whole blood, plasma, serum, or a tissue biopsy. In one embodiment, the tissue biopsy comprises a somatic cell. In one embodiment, the dengue fever infection comprises symptoms including but not limited to, headache, joint aches, muscle aches, nausea, swollen lymph nodes, and/or vomiting.

In one embodiment, the present invention contemplates a method, comprising: a) providing; i) a patient suspected of having a virus infection; ii) a biological sample derived from said patient, wherein said sample is capable of comprising a soluble interleukin 1 receptor-like 1 protein; and b) detecting said protein in said sample. In one embodiment, the virus includes, but is not limited to, flaviviruses and bunyaviruses. In one embodiment, the flavivirus includes, but is not limited to, a dengue virus, a yellow fever virus, a West Nile virus, and/or an encephalitis virus. In one embodiment, the bunyavirus includes, but is not limited to, a Hantaan virus and/or a Sin Nombre virus. In one embodiment, the detecting identifies that the protein is elevated to at least 1.5 times relative to other febrile illness. In one embodiment, the detecting comprises an antibody, wherein said antibody is directed to said protein. In one embodiment, the antibody is labeled. In one embodiment, the detecting comprises a primer, wherein said primer is complementary to a nucleic acid encoding said protein. In one embodiment, the identification of the elevated protein diagnoses said dengue fever infection.

In one embodiment, the present invention contemplates a kit, comprising: a) a reagent capable of detecting a soluble interleukin 1 receptor-like 1 protein; and b) a sheet of instructions capable of diagnosing a virus infection based upon said detected protein. In one embodiment, the instructions comprise dengue virus specific symptomology. In one embodiment, the instructions are capable of diagnosing a primary dengue virus infection. In one embodiment, the instructions are capable of diagnosing a secondary dengue virus infection. In one embodiment; the reagent comprises an antibody, wherein said antibody is directed to said protein. In one embodiment, the virus includes, but is not limited to, flaviviruses and bunyaviruses. In one embodiment, the flavivirus includes, but is not limited to, a dengue virus, a yellow fever virus, a West Nile virus, and/or an encephalitis virus. In one embodiment, the bunyavirus includes, but is not limited to, a Hantaan virus and/or a Sin Nombre virus. In one embodiment, the method further comprises a second sheet of instructions capable of diagnosing infection by non-viral hemorrhagic agents (i.e., for example, a bacterial infection).

In one embodiment, the present invention contemplates a kit, comprising: a) a reagent capable of detecting a soluble interleukin 1 receptor-like 1 nucleic acid; and b) a sheet of instructions capable of diagnosing a virus infection based upon said detected nucleic acid. In one embodiment, the instructions comprise dengue virus specific symptomology. In one embodiment, the instructions are capable of diagnosing a primary dengue virus infection. In one embodiment, the instructions are capable of diagnosing a secondary dengue virus infection. In one embodiment, the reagent comprises a primer, wherein said primer is complementary to said nucleic acid. In one embodiment, the virus includes, but is not limited to, flaviviruses and bunyaviruses. In one embodiment, the flavivirus includes, but is not limited to, a dengue virus, a yellow fever virus, a West Nile virus, and/or an encephalitis virus. In one embodiment, the bunyavirus includes, but is not limited to, a Hantaan virus and/or a Sin Nombre virus. In one embodiment, the method further comprises a second sheet of instructions capable of diagnosing infection by non-viral hemorrhagic agents (i.e., for example, a bacterial infection).

In one embodiment, the present invention contemplates a method, comprising: a) providing; i) a patient having an elevated soluble interleukin 1 receptor-like 1 protein and exhibiting at least one symptom of a virus infection; ii) a soluble interleukin 1 receptor-like 1 composition; and b) administering said composition to said patient under conditions such that said at least one symptom is reduced. In one embodiment, the virus includes, but is not limited to, flaviviruses and bunyaviruses. In one embodiment, the flavivirus includes, but is not limited to, a dengue virus, a yellow fever virus, a West Nile virus, and/or an encephalitis virus. In one embodiment, the bunyavirus includes, but is not limited to, a Hantaan virus and/or a Sin Nombre virus. In one embodiment, the composition comprises a soluble interleukin 1 receptor-like 1 polypeptide. In one embodiment, the composition comprises a soluble interleukin 1 receptor-like 1 mRNA. In one embodiment, the composition comprises a small molecule, wherein the molecule enhances soluble interleukin 1 receptor-like 1 mRNA expression. In one embodiment, the composition further comprises a liposome. In one embodiment, the administering comprises a topical administration. In one embodiment, the topical administration is selected from the group consisting of transdermal patches, ointments, lotions, creams, gels, drops, suppositories, sprays, liquids and powders. In one embodiment, the administering comprises parenteral administration. In one embodiment, the parenteral administration is selected from the group consisting of intravenous, intraarterial, subcutaneous, intraperitoneal or intramuscular injection or infusion; or intracranial, intrathecal or intraventricular, administration.

The present invention also provides a method of screening compounds, comprising providing a virus infected sample; and one or more test compounds; and contacting the virus infected sample with the test compound; and detecting a change in a sST2 composition expression in the virus infected sample in the presence of the test compound relative to the absence of the test compound. In one embodiment, the virus includes, but is not limited to, flaviviruses and bunyaviruses. In one embodiment, the flavivirus includes, but is not limited to, a dengue virus, a yellow fever virus, a West Nile virus, and/or an encephalitis virus. In one embodiment, the bunyavirus includes, but is not limited to, a Hantaan virus and/or a Sin Nombre virus. In some embodiments, the detecting comprises detecting sST2 mRNA. In other embodiments, the detecting comprises detecting sST2 polypeptide. In some embodiments, the sample comprises an in vitro cell. In other embodiments, the sample comprises an in vivo cell. In some embodiments, the test compound comprises a peptide. In other embodiments, the test compound comprises an oligonucleotide.

### DEFINITIONS

The term “interleukin-1 like receptor 1” or “IL1-LR1” refer to proteins which are capable of binding interleukin-33 (IL-33) molecules and, in their native configuration can act as a human plasma membrane protein, thereby playing a role in transducing a signal in the cell. Intact membrane receptors generally include an extracellular domain which binds to a ligand, a hydrophobic transmembrane domain which remains embedded within the plasma membrane lipid bilayer, and a cytoplasmic or intracellular domain which is believed to deliver a biological signal to effector cells via a cascade of chemical reactions within the cytoplasm of the cell. The hydrophobic transmembrane domain and a highly charged region of the cytoplasmic domain generally follows the transmembrane domain that cooperatively function to halt transport of the IL-1 receptor across the plasma membrane.

The term “soluble interleukin-1 like receptor 1” or “sIL1-LR1” or “sST2” means a polypeptide, or a substantially equivalent analog, having an amino acid sequence corresponding to the extracellular region of a native human IL-1 receptor or a polypeptide which varies from a native IL-1 receptor or polypeptide by one or more amino acid substitutions, deletions, or additions, and which retain the ability to bind IL-33. sST2 proteins lack a transmembrane region and are therefore secreted from cells through the plasma membrane.

The term “sST2 nucleotide sequence” as used herein, refers to any DNA sequence which codes for a soluble interleukin-1 like receptor 1 protein and may be made by constructing cDNAs which encode only the extracellular domain of an IL-1 receptor (i.e., for example, devoid of a transmembrane region) using various methods for DNA manipulation or mutagenesis. For example, cDNAs which encode sST2 may be constructed by truncating a cDNA encoding the full length IL-1 receptor 5′ of the transmembrane region, ligating synthetic oligonucleotides to regenerate truncated portions of the extracellular domain, if desired, and provide a stop codon to terminate transcription.

The term “isolated nucleic acid” as used herein, refers to any nucleic acid that is free of the nucleic acids that normally flank it in the genome. The term “nucleic acid” can encompass both RNA and DNA, and can include both naturally occurring and/or synthetic (e.g., chemically synthesized) nucleic acids.

The term “fusion protein” as used herein refers to a protein formed by expression of a hybrid gene made by combining two gene sequences. Typically this is accomplished by cloning a cDNA into an expression vector in frame with an existing gene. The fusion partner may act as a reporter (e.g., β-gal) or may provide a tool for isolation purposes (e.g., GST).

The term “gene” refers to a nucleic acid (e.g., DNA) sequence that comprises coding sequences necessary for the production of a polypeptide or precursor or RNA (e.g., tRNA, siRNA, rRNA, etc.). The polypeptide can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and the sequences located adjacent to the coding region on both the 5′ and 3′ ends, such that the gene corresponds to the length of the full-length mRNA. The sequences that are located 5′ of the coding region and which are present on the mRNA are referred to as 5′ untranslated sequences. The sequences that are located 3′ or downstream of the coding region and that are present on the mRNA are referred to as 3′ untranslated sequences. The term “gene” encompasses both cDNA and genomic forms of a gene. A genomic form or clone of a gene contains the coding region, which may be interrupted with non-coding sequences termed “introns” or “intervening regions” or “intervening sequences.” Introns are removed or “spliced out” from the nuclear or primary transcript, and are therefore absent in the messenger RNA (mRNA) transcript. The mRNA functions during translation to specify the sequence or order of amino acids in a nascent polypeptide.

As used herein, the term “purified” refers to molecules (polynucleotides or polypeptides) that are removed from their natural environment, isolated or separated.

The term “substantially pure”, as used herein, refers to a soluble interleukin-1 like receptor 1 composition free of other components of natural or endogenous origin and containing less than about 1% by mass of protein contaminants residual of production processes. “Substantially purified” molecules are at least 50% free, preferably at least 75% free, and more preferably at least 90% free from other components with which they are naturally associated.

Such compositions, however, can contain other proteins added as stabilizers, carriers, excipients or co-therapeutics.

The term “recombinant DNA” refers to a DNA molecule that is comprised of segments of DNA joined together by means of molecular biology techniques. Similarly, the term “recombinant protein” refers to a protein molecule that is expressed from recombinant DNA.

As used herein, the term “coding region” refers to the nucleotide sequences that encode the amino acid sequences found in the nascent polypeptide as a result of translation of an mRNA molecule. The coding region is bounded in eukaryotes, on the 5′ side by the nucleotide triplet “ATG” that encodes the initiator methionine and on the 3′ side by one of the three triplets which specify stop codons (i.e., for example, TAA, TAG, and TGA).

Where an amino acid sequence is recited herein to refer to an amino acid sequence of a naturally occurring protein molecule, “amino acid sequence” and like terms, such as “polypeptide” or “protein,” are not meant to limit the amino acid sequence to the complete, native amino acid sequence associated with the recited protein molecule. The term “wild-type” refers to a gene or gene product that has the characteristics of that gene or gene product when isolated from a naturally occurring source. A wild type gene is that which is most frequently observed in a population and is thus arbitrarily designed the “normal” or “wild-type” form of the gene.

In contrast, the terms “modified,” “mutant,” and “variant” refer to a gene or gene product that displays modifications in sequence and or functional properties (i.e., for example, altered characteristics) when compared to the wild-type gene or gene product. It is noted that naturally occurring mutants can be isolated wherein these may be identified by the fact that they have altered characteristics when compared to the wild-type gene or gene product.

The term “fragment” or “portion” when used in reference to a nucleotide sequence refers to that sequence, which ranges in size from 10 nucleotides to the entire nucleotide sequence minus one nucleotide. When used in reference to an amino acid sequence the term “fragment” or “portion” refers to that sequence, which ranges in size from 3 amino acids to the entire amino acid sequence minus one amino acid.

The terms “patient” or “subject” refer to a mammal or animal who is a candidate for receiving medical treatment. For example, a mammal may be a human.

As used herein, the term “effective amount” refers to the amount of a compound (e.g., an sST2 antagonist) sufficient to effect beneficial or desired results. An effective amount can be administered in one or more administrations, applications or dosages and is not limited intended to be limited to a particular formulation or administration route.

The term, “sST2 formulation” as used herein, refers to any compound and/or compounds capable of interfering with the synthesis, release, and/or activity of an sST2 protein, nucleotide, and/or gene. Such a composition may include, but not be limited to, a nucleic acid sequence (i.e., for example, sST2 mRNA), an amino acid sequence (i.e., for example, an sST2 polypeptide or fusion protein), or a small molecule (i.e, for example, any compound that may enhance sST2 mRNA expression).

As used herein, the term “therapeutic composition” refers to the combination of an active agent (i.e., for example, an sST2 antagonist) with a carrier, inert or active, making the composition especially suitable for diagnostic or therapeutic use in vivo, in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants. (See e.g., Martin, Remington's Pharmaceutical Sciences, 15th Ed., Mack Publ. Co., Easton, Pa. (1975)).

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. “Salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metals (e.g., sodium) hydroxides, alkaline earth metals (e.g., magnesium), hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄₊ (wherein W is a C₁₋₄ alkyl group), and the like. For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

As used herein, the terms “solid phase supports” or “solid supports,” are used in their broadest sense to refer to a variety of supports, some of which may be commercially available (infra). Solid phase supports include, but are not limited to, silica gels, resins, derivatized plastic films, glass beads, cotton, plastic beads, alumina gels, and the like. As used herein, “solid supports” also include synthetic antigen-presenting matrices, cells, liposomes, and the like. A suitable solid phase support may be selected on the basis of desired end use and suitability for various protocols. For example, for peptide synthesis, solid phase supports may refer to resins such as polystyrene (e.g., PAM-resin obtained from Bachem, Inc., Peninsula Laboratories, etc.), POLYHIPE) resin (obtained from Aminotech, Canada), polyamide resin (obtained from Peninsula Laboratories), polystyrene resin grafted with polyethylene glycol (TENTAGEL, Rapp Polymere, Tubingen, Germany) or polydimethylacrylamide resin (obtained from Milligen/Biosearch, California).

As used herein, the term “virus” refers to minute infectious agents, which with certain exceptions, are not observable by light microscopy, lack independent metabolism, and are able to replicate only within a living host cell. These individual particles (i.e., for example, virions) typically comprise nucleic acid and a protein shell or coat; some virions also have a lipid containing membrane. The term “virus” encompasses all types of viruses, including animal, plant, phage, and other viruses. In particular, a virus may refer to a flavivirus (i.e., for example, a dengue fever virus) and/or a bunyavirus (i.e., for example, a Hantaan virus).

The term “symptoms” as used herein, refers to any subjective evidence of a disease or physical disturbance observed by a patient.

The term “suspected of” as used herein, indicates that a subject or patient has been determined to be at increased risk, relative to the general population of such subjects or patients, of developing a particular disease or disorder, or symptom thereof, as herein defined. For example, a subject animal could have a personal and/or family medical history that includes frequent occurrences of a particular disease or disorder or be exposed to particular environmental circumstances known to result in an increased risk of exposure.

The term “administering” or “administer” as used herein, refers to providing a patient with a composition intended for therapeutic benefit. Such an administration may be parenteral or non-parenteral, acute, chronic, or under conditions such that a controlled release of a therapeutic composition takes place.

The term “topical” as used herein, refers to an administration to, or action on, any surface of a part of the body.

The term “parenteral” as used herein, refers to any administration of a therapeutic composition to a part of the body that does not involve the gastrointestinal system.

The term “map” as used herein, refers to any data complication reflecting the relative gene expression profiles of at least one gene marker. A map may comprises many gene markers such a 1-1,000 markers, preferably 100-500 markers, more preferably 200-300 markers.

The term “biomarker” as used herein, refers to any quantifiable biological component that is unique to a particular physiological condition (i.e., for example, a virus infection). For example, a biomarker may be mRNA resulting from transcription of a gene (i.e., for example, sST2 mRNA). Alternatively, a biomarker may be a protein resulting from translation of an mRNA (i.e., for example, sST2 polypeptide). A measurable increase or decrease, of a biomarker level, relative to a normal population, may provide a diagnosis of a particular physiological condition.

The term, “other febrile illness, as used herein, refers to any illness having a fever wherein genomic dengue RNA is not detectable, IgM antibodies are absent, and/or hemagglutination inhibition is increased <four-fold.

### BRIEF DESCRIPTION OF THE FIGURES

FIG. 1 presents exemplary data of a human microarray gene expression analysis identifying flavivirus and/or bunyavirus specific responses for fifty-one (51) selected gene transcripts. Signal values were calculated using the rma method (BioConductor/R; DFCI/Harvard Medical School). All virus infections were incubated for forty-eight hours in human umbilical vein endothelial cell cultures.—: untreated (n=4); C6: C6/36 cell culture control (n=1); D: dengue fever 2 virus (n=2); WN: West Nile virus (n=1); HN: Hantaan virus (n=2); SN: Sin Nombre virus (n=1); YF: Yellow Fever virus (n=1), V: Vacciniae virus (n=2); and EB: Epstein-Barr virus (n=1). Color indicates fold change from normalized median: dark red: ˜6 fold increase; dark blue: ˜6 fold decrease.

FIG. 2 presents a close-up view of the human microarray gene expression analysis shown in FIG. 1, for eleven (11) selected gene transcripts.

FIG. 3 presents exemplary data showing a human microarray gene expression analysis of eleven (11) selected gene transcripts following exposure to dengue fever virus in five different cell types. HUVECs (human umbilical vein endothelial cells); monocytes; CD4 T lymphocytes; CD8 T lymphocytes; and PBMCs (peripheral blood monocyte cells). Experimental conditions were performed in accordance with FIG. 1.

FIG. 4 presents exemplary data of soluble ST2 (sST2) protein levels in serum from OFI and dengue virus infected patients. Results are expressed as sST2 mean values (pg/ml)± standard error of mean for each patient group and each disease day. Mean sST2 levels for healthy donors was: 15.9±4.4 (N=14) pg/ml. Mann-Whitney statistical analysis between OFI and dengue virus infections at each disease day: significant differences between OFI and dengue at days −1 (p=0.0088) and day 0 (p=0.0004). Conval.: convalescence.

FIG. 5 presents exemplary data of soluble ST2 (sST2) protein levels in primary and secondary dengue virus infections. Results are expressed as sST2 mean values (pg/ml)± standard error of mean for each patient group and each disease day. Mean sST2 levels for healthy donors was: 15.9±4.4 (N=14) pg/ml. Mann-Whitney statistical analysis between primary and secondary dengue virus infections at each disease day: significant differences between primary and secondary infections at days −1 (p=0.0470) and day 0 (p=0.0300). Conval.: convalescence.

FIG. 6 presents one embodiment of a human interleukin 1 receptor-like 1 amino acid sequence (SEQ ID NO:1) (A) encoded by a nucleic acid sequence of SEQ ID NO:2 (B). Accession Number NM_{—}003856.

FIG. 7 presents one embodiment of a human interleukin 1 receptor like 1 homolog amino acid sequence (SEQ ID NO:3) (A) encoded by a nucleic acid sequence of SEQ ID NO:4 (B). Accession Number AK291578.

FIG. 8 presents one embodiment of a human interleukin 1 receptor-like 1 amino acid sequence (SEQ ID NO:5) (A) encoded by a nucleic acid sequence of SEQ ID NO:6 (B). Accession Number NM_{—}016232.

### DETAILED DESCRIPTION

This invention relates to the detection and diagnosis of inflammatory diseases. One such inflammatory disease is believed caused by the dengue fever virus. An infection caused by the dengue fever virus may result in conditions ranging from dengue fever, to dengue hemorraghic fever, to dengue septic shock. In one embodiment, the soluble ST2 protein is believed to act as a biomarker for dengue fever virus infection, and may be useful as a diagnostic kit for dengue virus.

Many viruses are relatively innocuous, such as the common cold. However, serious diseases may result from some virus infections. For example, twelve distinct viruses associated with hemorrhagic fever in humans are classified among four families: Arenaviridae, which includes Lassa, Junin, and Machupo viruses; Bunyaviridae, which includes, but are not limited to, Rift Valley fever, Crimean-Congo hemorrhagic fever, and Hantaan viruses; Filoviridae, which includes Marburg and Ebola viruses; and Flaviviridae, which includes but is not limited to, yellow fever, dengue, Kyasanur Forest disease, and Omsk viruses. Most hemorrhagic fever viruses are zoonoses, with the possible exception of the four dengue viruses, which may continually circulate among humans. Hemorrhagic fever viruses are found in both temperate and tropical habitats and generally infect both sexes and all ages, although the age and sex of those infected are frequently influenced by the possibility of occupational exposure. Transmission to humans is frequently by bite of an infected tick or mosquito or via aerosol from infected rodent hosts. Aerosol and nosocomial transmission are especially important with Lassa, Junin, Machupo, Crimean-Congo hemorrhagic fever, Marburg, and Ebola viruses. Seasonality of hemorrhagic fever among humans is influenced for the most part by the dynamics of infected arthropod or vertebrate hosts. Mammals, especially rodents, appear to be important natural hosts for many hemorrhagic fever viruses. The transmission cycle for each hemorrhagic fever virus is distinct and is dependent upon the characteristics of the primary vector species and the possibility for its contact with humans. LeDuc J W, “Epidemiology of hemorrhagic fever viruses” Rev Infect Dis. 11:S730-S735 (1989).

### I. Dengue Fever

Symptoms of dengue fever (DF) may include, but are not limited to, high fever, headache, myalgias, skin rash, thrombocytopenia, coagulation alterations, hepatic inflammation and hemorrhagic manifestations. Increased vascular permeability that results in vascular leakage is the characteristic event that occurs and defines dengue hemorrhagic fever (DHF). Rothman et al., “Immunopathogenesis of Dengue hemorrhagic fever” Virology 257:1-6 (1999).

Dengue virus can be classified into four antigenically distinct serotypes: D1V, D2V, D3V, and D4V, and each one of them can cause DF or DHF. Monath T. P., “Dengue: the risk to developed and developing countries” Proc Natl Acad Sci USA; 91:2395-2400 (1994). Infection with one of the serotypes imparts immunity to the infecting serotype. Multiple infections with different (heterologous) serotypes can occur during one's lifetime and DHF/DSS is usually associated with secondary infections. Halstead et al., “Observations related to pathogenesis of dengue hemorrhagic fever. IV. Relation of disease severity to antibody response and virus recovered” Yale J Biol Med 42:311-28 (1970); and Guzman et al., “Dengue hemorrhagic fever in Cuba 1981: a retrospective seroepidemiologic study” Am J Trop Med Hyg 42:179-184 (1990). When a secondary infection occurs, the immune response could be dominated by the pre-existing cross-reactive memory cells from a previous dengue infection rather than by the naive pool of high-affinity specific cells for the infecting serotype, sometimes referred to as an ‘original antigenic sin’. Rothman A. L., “Dengue: defining protective versus pathologic immunity” J Clin Invest 113:946-951 (2004). These low-affinity memory clones are rapidly activated and undergo clonal expansion. This results in the production of antibodies that bind to the heterologous serotype at non-neutralizing epitopes, which could lead to antibody-mediated immune enhancement instead of blocking viral infectivity. Halstead S. B., “Antibody, macrophages, dengue virus infection, shock, and hemorrhage: a pathogenetic cascade” Rev Infect Dis; 11 Suppl 4: S830-839 (1989); and Morens D. M., “Antibody-dependent enhancement of infection and the pathogenesis of viral disease” Clin Infect Dis 19:500-512 (1994). Cross reactivity also generates a dysfunctional T cell response that results in suboptimal clearance of the virus and an uncontrolled production of soluble mediators. Welsh et al., “Dengue immune response: low affinity, high febrility” Nat Med 9:820-822 (2003).

### II. The Interleukin-1 Receptor Like-1 Protein (ST2)

Interleukin-1α and interleukin-1β (IL-1α and IL-1β) are distantly related polypeptide cytokines which are believed to play a role in the regulation of immune and inflammatory responses. These two proteins were originally both classified as IL-1 comprising a shared lymphocyte activation factor (LAF) activity and a common major cellular source (i.e., for example, activated macrophages). Studies using purified natural and recombinant IL-1 molecules suggest that IL-1α and IL-1β may mediate specific activities previously ascribed to IL-1.

IL-1α and IL-1β mediate their biological activities via at least two classes of plasma membrane bound receptors. One of these classes of receptor is expressed primarily on T cells and fibrobrasts. IL-1α and IL-1β bind to this class of IL-1 receptor, resulting in transduction of a biological signal to various immune effector cells. Because mature full-length IL-1 receptors are bound to the plasma membrane, however, they cannot be effectively used in assay, diagnosis or therapy to regulate immune or inflammatory activities. Dower, et al., “A soluble form of a human IL-1 receptor protein has been described as useful in treating inflammatory diseases resulting from elevated IL-1 levels” U.S. Pat. No. 5,488,032 (herein incorporated by reference).

The Interleukin-1 receptor like-1 protein (IL-1RL-1 or ST2) is a member of the interleukin-1 receptor (IL-1R) family of proteins. sST2 has been reported as: i) a primary response gene for murine fibroblasts (Tominaga S., “A putative protein of a growth specific cDNA from BALB/c-3T3 cells is highly similar to the extracellular portion of mouse interleukin 1 receptor” FEBS Lett 258:301-304 (1989); and Yanagisawa et al., “Murine ST2 gene is a member of the primary response gene family induced by growth factors” FEBS Lett 302:51-53 (1992); and ii) an HA-ras oncogen-responsive gene (Werenskiold et al., “Induction of a mitogen-responsive gene after expression of the Ha-ras oncogene in NIH 3T3 fibroblasts” Mol Cell Biol 9:5207-5214 (1989).

Alternative splicing of the ST2 gene is believed to generate at least three mRNAs; i) ST2L, corresponding to a longer membrane-anchored form; ii) sST2, a shorter released soluble form; and iii) ST2V, a membrane bound variant form. Yanagisawa et al., “Presence of a novel primary response gene ST2L, encoding a product highly similar to the interleukin 1 receptor type 1” FEBS Lett 318: 83-87 (1993); Bergers et al., “Alternative promoter usage of the Fos-responsive gene Fit-1 generates mRNA isoforms coding for either secreted or membrane-bound proteins related to the IL-1 receptor” Embo J 13:1176-1188 (1994); and Tominaga et al., “Presence and expression of a novel variant form of ST2 gene product in human leukemic cell line UT-7/GM” Biochem Biophys Res Commun 264:14-18 (1999). The expression of the three forms has been detected in various human tissues and cells, including hematopoietic and endothelial cells. Kumar et al., “Expression of ST2, an interleukin-1 receptor homologue, is induced by proinflammatory stimuli” Biochem Biophys Res Commun 235: 474-478 (1997).

ST2L has been reported to be selectively expressed on Th2 CD4+ T cells, but not on Th1 CD4+ T cells, and therefore is proposed as a biomarker for Th2 CD4+ T cells. Yanagisawa et al., “The expression of ST2 gene in helper T cells and the binding of ST2 protein to myeloma-derived RPMI8226 cells” J Biochem (Tokyo; 121:95-103 (1997); and Xu et al., “Selective expression of a stable cell surface molecule on type 2 but not type 1 helper T cells” J Exp Med 187:787-794 (1998). Alternatively, ST2L might also be involved in the effector phase of Th2 immune responses. Trajkovic et al., “T1/ST2—an IL-1 receptor-like modulator of immune responses” Cytokine Growth Factor Rev 15:87-95 (2004).

Expression of sST2 protein has been reported to be induced in vitro by pro-inflammatory stimuli including lipo-polysaccaride (LPS), IL-1β, and TNF-α and IL-6 in human and murine inflammatory models. Kumar et al., “Expression of ST2, an interleukin-1 receptor homologue, is induced by proinflammatory stimuli” Biochem Biophys Res Commun 235: 474-478 (1997); and, Tajima et al., “The increase in serum soluble ST2 protein upon acute exacerbation of idiopathic pulmonary fibrosis” Chest 124:1206-1214 (2003). In one mouse model, proinflammatory cytokine production precedes sST2 expression. Oshikawa et al., “ST2 protein induced by inflammatory stimuli can modulate acute lung inflammation” Biochem Biophys Res Commun 299:18-24 (2002). Elevated levels of sST2 have been found in patients with inflammatory disorders associated with abnormal Th2 mediated responses, including: i) autoimmune diseases (Kuroiwa et al., “Identification of human ST2 protein in the sera of patients with autoimmune diseases” Biochem Biophys Res Commun 284:1104-1108 (2001); ii) asthma (Oshikawa et al., “Elevated soluble ST2 protein levels in sera of patients with asthma with an acute exacerbation” Am J Respir Crit Care Med 164:277-281 (2001); and Oshikawa et al., “Expression and function of the ST2 gene in a murine model of allergic airway inflammation” Clin Exp Allergy 32:1520-1526 (2002); iii) idiopathic pulmonary fibrosis (Tajima et al., “The increase in serum soluble ST2 protein upon acute exacerbation of idiopathic pulmonary fibrosis” Chest 124:1206-1214 (2003); and iv) sepsis (Brunner et al., “Increased levels of soluble ST2 protein and IgG1 production in patients with sepsis and trauma” Intensive Care Med 30:1468-1473 (2004).

Further, sST2 levels have also been reported as elevated in patients with other inflammatory conditions, like LPS induced inflammation and myocardial infarction. Oshikawa et al., “Expression of ST2 in helper T lymphocytes of malignant pleural effusions” Am J Respir Crit Care Med 165:1005-1009 (2002); and Shimpo et al., “Serum levels of the interleukin-1 receptor family member ST2 predict mortality and clinical outcome in acute myocardial infarction” Circulation 109:2186-2190 (2004). sST2 has also been proposed as a biomarker for heart failure. Weinberg et al., “Identification of serum soluble ST2 receptor as a novel heart failure biomarker” Circulation 107: 721-726 (2003).

### III. Clinical Detection of Elevated Cytokine Levels in Dengue Fever

Dengue virus infection is believed to be an acute infection which may involve an over-production of pro-inflammatory molecules. In one embodiment, the present invention contemplates diagnosing dengue fever infected patients by detecting elevated sST2 protein levels in blood (i.e., for example, whole blood, blood plasma, and/or blood serum). In one embodiment, a secondary dengue virus infection comprises a higher serum sST2 protein level as compared to primary dengue virus infection.

A. Non-sST2 Cytokines

Elevated circulating levels of both type 1 (Th1) and type 2 (Th2) cytokines and various chemokines including gamma interferon (IFN-γ), tumor necrosis factor alpha (TNF-α), interleukin (IL)-1beta (IL-1β), IL-6, IL-10, IL-13, IL-8, macrophage chemoattractant protein-1 (MCP-1), and macrophage inflammatory protein 1 beta (MIP-1β) have been detected in dengue infected patients, and the kinetics and persistence of some of these mediators seems to be related to the severity of the disease. Hober et al., “Serum levels of tumor necrosis factor-alpha (TNF patients” Am J Trop Med Hyg 48:324-331 (1993); Hober et al., “High levels of sTNFR p75 and TNF alpha in dengue-infected patients” Microbiol Immunol; 40:569-573 (1996); Raghupathy et al., “Elevated levels of IL-8 in dengue hemorrhagic fever” J Med Virol 56:280-5 (1998); Green et al., “Elevated plasma interleukin-10 levels in acute dengue correlate with disease severity” J Med Virol 59:329-334 (1999); Mustafa et al., “Elevated levels of interleukin-13 and IL-18 in patients with dengue hemorrhagic fever” FEMS Immunol Med Microbiol 30:229-233 (2001); Spain-Santana et al., “MIP-1 alpha and MIP-1 beta induction by dengue virus” J Med Virol 65: 324-330 (2001); Libraty et al., “Differing influences of virus burden and immune activation on disease severity in secondary dengue-3 virus infections” J Infect Dis 185:1213-1221 (2002); Avila-Aguero et al., “Systemic host inflammatory and coagulation response in the Dengue virus primo-infection” Cytokine 27:173-179 (2004); and Lee et al., “MCP-1, a highly expressed chemokine in dengue haemorrhagic fever/dengue shock syndrome patients, may cause permeability change, possibly through reduced tight junctions of vascular endothelium cells” J Gen Virol 87:3623-3630 (2006).

B. sST2 Cytokines

In one embodiment, the present invention contemplates a biomarker for an inflammatory response, wherein the biomarker comprises a soluble form of an interleukin-1 receptor like 1 protein (i.e., for example, IL-1RL-1 and/or sST2). In one embodiment, the biomarker levels are elevated in patients with diseases characterized by the inflammatory response. In one embodiment, the inflammatory response comprises dengue fever. In one embodiment, the inflammatory response comprises dengue hemorrhagic fever. In one embodiment, the present invention contemplates a biomarker for a secondary dengue virus infection. In one embodiment, the inflammatory response comprises dengue fever septic shock.

1. sST2 Expression in Microarrays

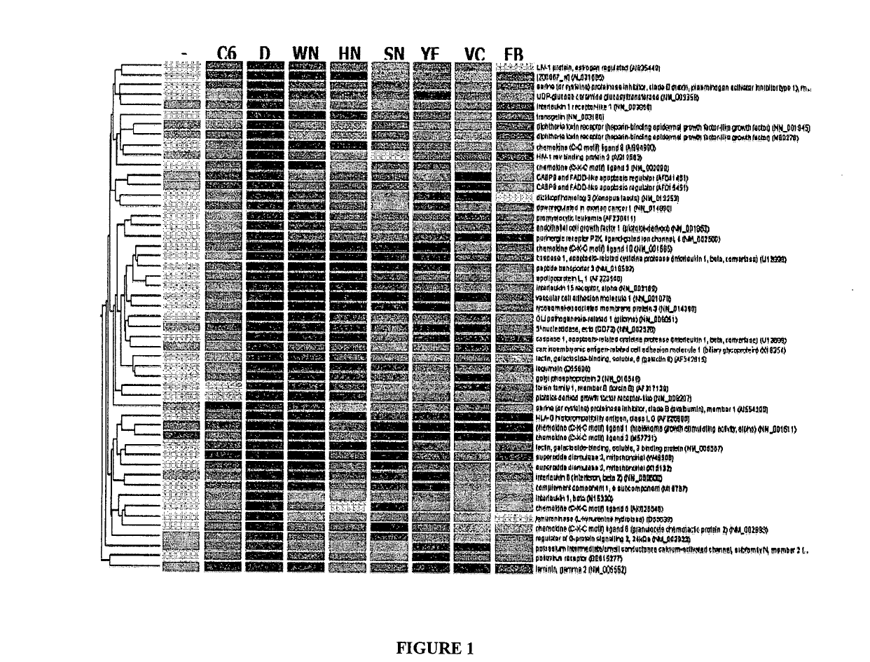

Affymetrix HG-U133A GeneChips® microarrays containing 22,283 human transcripts were used to screen for flavivirus and/or bunyavirus specific gene expression in human umbilical vein endothelial cell cultures (HUVECs). See, FIG. 1. Dengue fever D2V virus (D) specific gene expression was identified by comparison to other flaviviruses and/or bunyaviruses including, but not limited to, West Nile virus (WN), Hantaan virus (HN), Sin Nombre virus (SN), Yellow Fever virus (YF), Vaccinea virus (VC), and Epstein-Barr virus (EB). Each array was median normalized, wherein the genes were normalized to the median of untreated HUVECs. All 22,283 human gene transcripts were analyzed by 1-way analysis of variance (ANOVA) to identify genes with statistically significant differences between the two groups: −D2V and +D2V. ANOVA statistics were calculated using a parametric cross-gene error model with a p-value cutoff of 0.01 having a repeated measures (i.e., multiple testing) correction (i.e., for example, the Benjamini and Hochberg False Discovery Rate). Under these conditions, approximately 1% of the 51 identified genes would be expected to pass the restriction by chance (Gene Spring®, Aligent). Hierarchical cluster analyses were also performed using a Pearson correlation (left-side brackets).

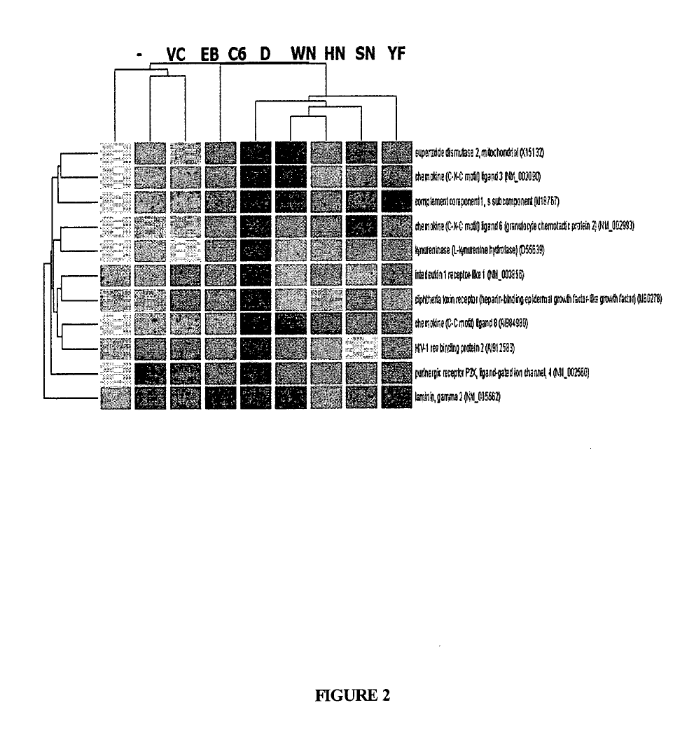

Eleven genes were identified as being differentially expressed in response to a dengue virus fever infection. See, FIG. 2. In particular, interleukin 1 receptor-like 1 (NM_{—}003856) appeared to be preferentially expressed, even when compared to other closely related flaviviruses. While other flaviviruses show some interleukin 1 receptor-like 1 protein expression, a lower level of induced sST2 mRNA expression by other flavivirus and/or bunyavirus is seen by the variations in red color intensity when compared to the dengue fever virus: D>YF>HN=VC>WN. Other flavivirus and/or bunyavirus demonstrated sST2 mRNA expression at/or below controls, as indicated by the variations in blue color intensity: SN=control>C6>EB. These observations indicated that interleukin 1 receptor-like 1 protein (IL1-RL1 or sST2) might be useful as a biomarker for flavivirus and/or bunyvirus infection (i.e., for example, dengue fever virus infection).

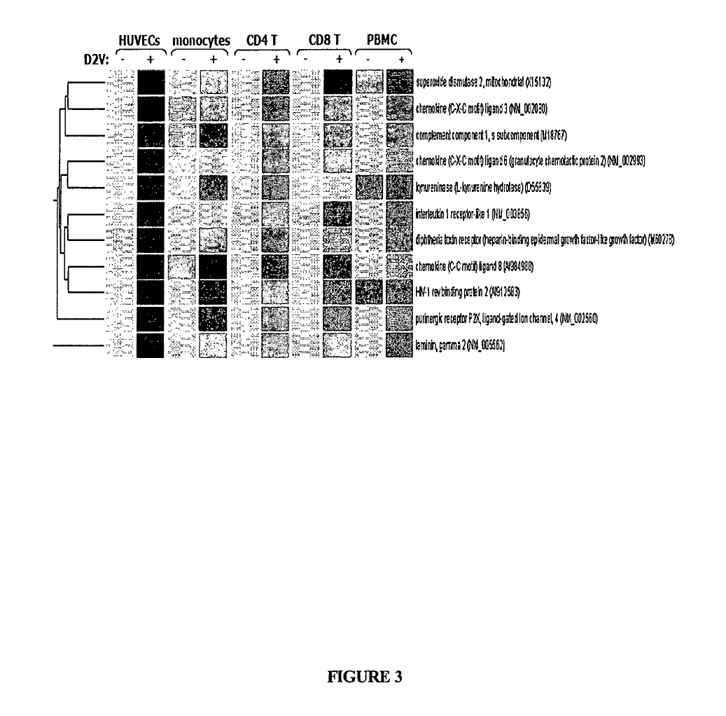

In one embodiment, the present invention contemplates a specific expression of sST2 mRNA in response to dengue fever virus infection in somatic cell expression. Although it is not necessary to understand the mechanism of an invention, it is believed that sST2 mRNA expression is not observed in cells responsible for an overall inflammatory response to infection. For example, following a forty-eight (48) hour dengue fever virus incubation, sST2 mRNA expression was observed only in HUVECs and not in monocytes, CD4 T or CD8 T lymphocytes, or peripheral blood mononuclear cells (PBMCs). See, FIG. 3. These data is further understood by noting that CD4 and CD8 T cells are not susceptible to dengue virus infection, consequently any observed sST2 induction CD4 and CD8 T cells would most like result from a non-specific induction due to the generalized inflammatory response.

2. Clinical Detection of sST2 Protein

Twenty-four (24) patients with confirmed dengue fever virus infection, classified as dengue fever, and eleven (11) patients with Other Febrile Illness (OFI) were evaluated. Dengue fever infected patients had serum sST2 protein levels elevated at least 1.5 times as compared to OFI patients both at the end of the febrile stage and at defervescence (p=0.0088 and p=0.0004 respectively). Further, patients with secondary dengue virus infections had serum sST2 protein levels elevated at least 1.5 times as compared with patients with primary dengue virus infections (p=0.047 at last day of fever and p=0.030 at defervescence). Furthermore, in dengue virus infected patients, a significant negative correlation was found between sST2 protein levels and platelet counts, but a positive correlation was found between thrombin time and transaminase activity.

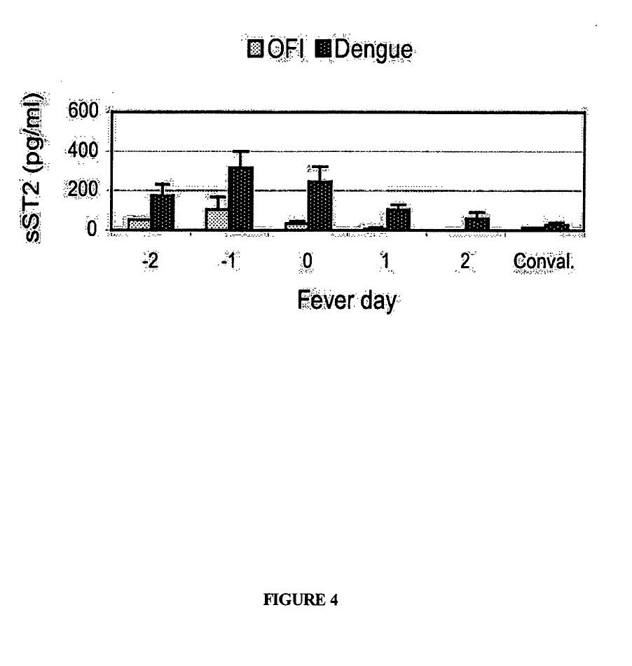

The data presented herein shows higher levels of sST2 protein in serum from dengue virus infected patients as compared to Other Febrile Illness (OFI) patients (i.e., for example, patients not positive for dengue fever virus specific IgM and/or genomic RNA). For example, serum levels of sST2 protein were found to be elevated at the end of the febrile stage of the disease, reaching a peak between fever days −1 and 0 followed by a decrease of the levels to normal values in convalescence. See, FIG. 4. Maximum sST2 levels also correlated to the final antibody titer, as higher levels of sST2 protein were found in patients who had higher HI titer, an indicator of secondary infections.

Further, a correlational analysis of sST2 protein levels was performed against certain laboratory parameters associated with dengue fever severity. For example, dengue virus infections are characterized by: i) thrombocytopenia (Srichaikul et al., “Haematology in dengue and dengue haemorrhagic fever” Baillieres Best Pract Res Clin Haematol 13: 261-276 (2000) ii) prolonged thrombin time (Krishnamurti et al., “Mechanisms of hemorrhage in dengue without circulatory collapse” Am J Trop Med Hyg 65:840-847 (2001); and Sosothikul et al., “Activation of endothelial cells, coagulation and fibrinolysis in children with Dengue virus infection” Thromb Haemost 97:627-34 (2007); and iii) elevated hepatic transaminase activity (Kalayanarooj et al., “Early clinical and laboratory indicators of acute dengue illness” J Infect Dis 176:313-321 (1997). The correlation analysis demonstrated that sST2 had a negative correlation with platelet count (i.e., for example, sST2 protein levels were higher when platelet counts were lower) and a positive correlation with both prolonged thrombin time (i.e., for example, sST2 levels were higher when thrombin time was longer) and AST/ALT activity (i.e., for example, sST2 levels were higher in patients with higher AST/ALT activity). See, Example 7.

Although it is not necessary to understand the mechanism of an invention, it is believed that peripheral blood mononuclear cells from dengue infected patients may be responsible for sST2 protein production as preliminary results using quantitative RT-PCR (Low Density Arrays) have shown an increased expression of ST2 mRNA (data not shown). It is further believed that sST2 protein could be involved in the inflammatory response as well as in Th2 immune responses. Amatucci et al., “Recombinant ST2 boosts hepatic Th2 response in vivo” J Leukoc Biol (2007); and Tajima et al., “ST2 gene induced by type 2 helper T cell (Th2) and proinflammatory cytokine stimuli may modulate lung injury and fibrosis” Exp Lung Res 33:81-97 (2007).

Some evidence suggests that sST2 could act as an anti-inflammatory mediator, through mechanisms involving: i) the inhibition of Toll-like receptor signaling by sequestration of MyD88 and Mal adapter proteins (Sweet et al., “A novel pathway regulating lipopolysaccharide-induced shock by ST2/T1 via inhibition of Toll-like receptor 4 expression” J Immunol 166: 6633-6639 (2001); and Brint et al., “ST2 is an inhibitor of interleukin 1 receptor and Toll-like receptor 4 signaling and maintains endotoxin tolerance” Nat Immunol 5:373-379 (2004)); or ii) inhibition of I-κB degradation resulting in down-regulation of NF-κB. Takezako et al., “ST2 suppresses IL-6 production via the inhibition of IkappaB degradation induced by the LPS signal in THP-1 cells” Biochem Biophys Res Commun 341:425-32 (2006).

In vitro and in vivo experiments have shown that sST2 protein, or an ST2-fusion protein, is able to attenuate the production of pro-inflammatory cytokines IL-1β, TNF-α, IL-6, and IL-12. Oshikawa et al., “ST2 protein induced by inflammatory stimuli can modulate acute lung inflammation” Biochem Biophys Res Commun 299:18-24 (2002); Sweet et al., “A novel pathway regulating lipopolysaccharide-induced shock by ST2/T1 via inhibition of Toll-like receptor 4 expression” J Immunol 166:6633-6639 (2001); and Leung et al., “A novel therapy of murine collagen-induced arthritis with soluble T1/ST2” J Immunol 173:145-150 (2004). Further, in two mouse models of ischemia/reperfusion, pre-treatment with an sST2-Fc fusion protein decreased the inflammatory response. Yin et al., “Pretreatment with soluble ST2 reduces warm hepatic ischemia/reperfusion injury” Biochem Biophys Res Commun 351:940-946 (2006): and Fagundes et al., “ST2, an IL-1R family member, attenuates inflammation and lethality after intestinal ischemia and reperfusion” J Leukoc Biol 81:492-499 (2007). Some evidence suggests IL-10 as a possible mediator of inflammatory responses. Fagundes et al., “ST2, an IL-1R family member, attenuates inflammation and lethality after intestinal ischemia and reperfusion” J Leukoc Biol 81:492-499 (2007). Other evidence indicates that high levels of pro-inflammatory cytokines like TNF-α and IL-6 have been found in dengue patients. Hober et al., “Serum levels of tumor necrosis factor-alpha (TNFalpha), interleukin-6 (IL-6), and interleukin-1 beta (IL-1 beta) in dengue-infected patients” Am J Trop Med Hyg 48:324-31 (1993); Hober et al., “High levels of sTNFR p75 and TNF alpha in dengue-infected patients” Microbiol Immunol 40:569-73 (1996); and Avila-Aguero et al., “Systemic host inflammatory and coagulation response in the Dengue virus primo-infection” Cytokine 27:173-179 (2004). Others have suggested that these cytokines might induce synthesis and/or release of an sST2 protein. Kumar et al., “Expression of ST2, an interleukin-1 receptor homologue, is induced by proinflammatory stimuli” Biochem Biophys Res Commun 235:474-8 (1997); and Tajima et al., “The increase in serum soluble ST2 protein upon acute exacerbation of idiopathic pulmonary fibrosis” Chest 124:1206-1214 (2003).

Although it is not necessary to understand the mechanism of an invention, it is believed that elevated sST2 protein levels found in dengue fever virus patients could be an indication of immune hyperactivation and/or a mechanism to down-regulate inflammation. For example, other evidence suggests that sST2 protein could act as a negative regulator of the Th2 response. Amatucci et al., “Recombinant ST2 boosts hepatic Th2 response in vivo” J Leukoc Biol (2007); and Schmitz et al., “IL-33, an interleukin-1-like cytokine that signals via the IL-1 receptor-related protein ST2 and induces T helper type 2-associated cytokines” Immunity 23:479-490 (2005). Recently, it has been suggested that sST2 could be acting as a decoy receptor for IL-33 regulating its biological function. In myocardium, IL-33/ST2L interactions are reported as cardioprotective and sST2 seems to have a role blocking anti-hypertrophic effect of IL-33. Sanada et al., “IL-33 and ST2 comprise a critical biomechanically induced and cardioprotective signaling system” J Clin Invest 117:1538-1549 (2007). IL-33 was identified as a ligand for ST2L, a marker of Th2 T lymphocytes. Schmitz et al., “IL-33, an interleukin-1-like cytokine that signals via the IL-1 receptor-related protein ST2 and induces T helper type 2-associated cytokines” Immunity 23:479-490 (2005). ST2L may be involved in the regulation of the Th2 associated immune response at the effector stage and in Th2 driven immunopathology. For example, the interaction of IL-33 with ST2L might lead to the induction of the Th2 cytokines IL-4, IL-5 and IL-13 through a signaling mechanism that could involve the activation of NF-κB and MAP kinases.

In dengue virus infections, a shift from a predominant Th1 response to a Th2 response around the time of defervescence appears to correlate with disease severity. Mustafa et al., “Elevated levels of interleukin-13 and IL-18 in patients with dengue hemorrhagic fever” FEMS Immunol Med Microbiol 30:229-233 (2001); and Chaturvedi et al., “Cytokine cascade in dengue hemorrhagic fever: implications for pathogenesis” FEMS Immunol Med Microbiol 28:183-188 (2000). Further, higher levels of IL-10 and IL-13 have been found in DHF compared to DF patients. Green et al., “Elevated plasma interleukin-10 levels in acute dengue correlate with disease severity” J Med Virol 59:329-334 (1999); Mustafa et al., “Elevated levels of interleukin-13 and IL-18 in patients with dengue hemorrhagic fever” FEMS Immunol Med Microbiol; 30:229-233 (2001); and Chen et al., “Altered T helper 1 reaction but not increase of virus load in patients with dengue hemorrhagic fever” FEMS Immunol Med Microbiol 44:43-50 (2005). Although it is not necessary to understand the mechanism of an invention, it is believed that elevated levels of sST2 protein could be part of a down-regulation mechanism triggered to attenuate the Th2 response that occurs in dengue patients. In one embodiment, the present invention contemplates a method comprising treating a virus infection (i.e., for example, a dengue fever virus infection) by administering an sST2 polypeptide and/or sST2 fusion protein.

Overall, the data presented herein show a transient elevation of sST2 protein levels in the serum of dengue virus-infected patients around the time of defervescence. Additionally, higher sST2 protein levels correlate with other biomarkers of severity in dengue virus infections. Supporting frequent observations that severe dengue fever manifestations correlate with secondary infections, these results show that levels of sST2 protein in serum were not only higher in patients with secondary infections, but also in patients with more severe manifestations.

### IV. Diagnostic Dengue Fever Kits

There are several diagnostic kits commercially available that are related to the diagnosis and detection of dengue fever virus infections. Such kits are usually designed to detect circulating antibodies directed to dengue fever virus antigens generated by the infected patient. Monath et al., “Diagnosis of flavivirus infection” U.S. Pat. No. 6,682,883 (herein incorporated by reference); and Groen et al., “Evaluation of six immunoassays for detection of dengue virus-specific immunoglobulin M and G antibodies” Clin Diagn Lab Immunol 7:867-871 (2000). Other available kits utilize labeled antibodies directed to specific antigens on the dengue virus. Chan et al., “Dengue virus peptides and methods” U.S. Pat. No. 5,824,506 (herein incorporated by reference). Several available kits are listed in Table 1 below.

| Company | Assay type |
|---|---|
| Biorad | NS1 dengue virus antigen detection by ELISA |
| Chemicon | Detecting IgM to dengue |
| MRL diagnostics | Detecting IgM to dengue |
| Veredus (singapore) | PCR detection |
| Calbiotech | Detecting igG to Dengue |
| Bioquant | 1) Detecting IgM to dengue |
| | 2) Detecting igG to Dengue |
| PanBio (Australia) | 1) Detecting IgM to dengue |
| | 2) Detecting igG to Dengue |
| Progen Biotechnik | 1) Detecting IgM to dengue |
| | 2) Detecting IgG to Dengue |
| INDX Integrated | 1) Detecting IgM to dengue (dipstick ELISA) |
| diagnostics, Baltimore, | 2) Detecting igG to Dengue (dipstick ELISA) |
| MD, USA | |
| Genelabs Diagnostics | 1) Detecting IgM to dengue (blot) |
| (singapore) | 2) Detecting igG to Dengue (blot) |

None of these kits utilize or contemplate the use of antibodies to detect an sST2 protein and/or sST2 nucleic acid derived from a biological sample as a biomarker of a dengue fever virus infection.

In one embodiment, the present invention provides kits for the detection and characterization of a virus infection (i.e., for example, a dengue fever virus infection). In some embodiments, the kit contains at least one antibody directed to a protein expressed as a result of dengue virus infection, in addition to detection reagents and buffers. In other embodiments, the kit may contain reagents capable of detecting mRNA or cDNA (e.g., oligonucleotide probes or primers) encoding a protein derived from a biological sample expressed as a result of the virus infection. In preferred embodiments, the kits contain all of the components necessary to perform a detection assay, including all controls, directions for performing assays, and any necessary software for analysis and presentation of results.

In another embodiment, the present invention contemplates kits for the practice of the methods of this invention. The kits preferably include one or more containers containing a protein and/or DNA detection method of this invention. The kit can optionally include a non-dengue virus infected biological sample to be utilized as a control. The kit can optionally include nucleic acids capable of hybridizing to a gene region specifically expressed in response to a dengue fever infection (i.e., for example, PCR primers specific to an sST2 gene region). The kit can optionally include enzymes capable of performing PCR (i.e., for example, DNA polymerase, Taq polymerase and/or restriction enzymes). The reagents may be provided suspended in the excipient and/or delivery vehicle or may be provided as a separate component which can be later combined with the excipient and/or delivery vehicle.

The kits may also optionally include appropriate systems (e.g. opaque containers) or stabilizers (e.g. antioxidants) to prevent degradation of the reagents by light or other adverse conditions.

The kits may optionally include instructional materials containing directions (i.e., protocols) providing for the use of the reagents in the diagnosis and/or detection of dengue fever infections within a patient. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

Kits useful in the methods contemplated herein are capable of detecting sST2 protein and/or their respective encoding nucleic acids, which are described herein as a biomarker for determining the severity of dengue virus infections. It would be expected that such kits be able to determine such features including, but not limited to, i) host response to dengue infection; ii) severity of dengue infection; iii) detection of secondary dengue virus infection; and iv) diagnose dengue fever related conditions and their associated secondary complications.

A. Antibody Kits

The detection of circulating sST2 protein in human patients may be accomplished by a commercially available ELISA kit (i.e., for example, ST2 ELSA Kit, Molecular and Biological Laboratory Co, Ltd., Woburn, Mass.). Tominaga et al., “Monoclonal antibody and method and kit for immunoassay of soluble human ST2” U.S. Pat. No. 7,087,396 (herein incorporated by reference). In one embodiment, the present invention contemplates a method for determining a soluble sST2 protein in a sample derived from a patient (i.e., for example, a human patient) expressing at least one symptom of a dengue fever virus infection. In one embodiment, the method comprises contacting a biological sample from a dengue fever infected patient with an immobilized antibody, wherein antibody is directed towards an sST2 protein. In one embodiment, the contacting comprises attaching a first sST2 antibody to a solid support, wherein antibody binds to a first epitope on the sST2 protein to create a first reaction product. In one embodiment, the first reaction product is reacted with a labeled second sST2 antibody, wherein the second antibody binds to a second epitope on the sST2 protein. In one embodiment, the method further comprises determining the amount of the label on the first reaction product, wherein the soluble sST2 protein is detected. In addition, a recombinant sST2 protein may be employed as a standard to prepare a calibration curve, based on which the ST2 in a sample is quantified.

Exemplary sources of useful antibodies that are capable of detecting sST2 are provided in Table 2.

TABLE 2
Sources Of ST2 Antibody

| Company | Antibody |
|---|---|
| MBL International | Mouse anti Human ST2 Monoclonal, Clone 2A5 |
| MBL International | Mouse anti Human ST2 Monoclonal, Clone FB9 |
| MBL International | Mouse anti Human ST@ Monoclonal, Clone HB12 |
| MD Biosciences | Mouse anti Human ST@ Monoclonal, Clone B4E6 |
| R&D Systems | Goat anti Humans IL1R4/ST2 polyclonal antibody |
| R&D Systems | Mouse anti Human IL-1 R4/ST2 Monoclonal, |
| | Clone 97203 |
| Santa Cruz | Goat anti (ST2(C-20) polyclonal antibody |
| Biotechnology, Inc. | |
| BIODESIGN & | Mouse anti Human IL-1R Monoclonal |
| OEM Concepts of | antibody Clone BD1204 |
| Meridian Life | |
| Science, Inc. | |
| Lifespan | Mouse anti Human IL-1R Monoclonal antibody |
| Biosciences | cat#LS-C16025 |
| Lifespan | Interleukin 1 Receptor, type 1(1I1R1) |
| Biosciences | cat# LS-C24843 |
| Novus Biologicals | Rabbit anti IL-1R1 monoclonal clone, EP409Y |

B. Nucleotide Kits

In one embodiment, the present invention contemplates a method to detect sST2 nucleotide sequences using specific primers which amplify a portion of the sST2 gene that can be used in a rapid reverse transcriptase-polymerase chain reaction (RT-PCR) method for specific detection of dengue viruses, but not other flaviviruses, such as West Nile virus, Japanese encephalitis virus and yellow fever virus, or the alphavirus Sindbis virus. The method enables diagnosis of dengue virus infection within six hours.

In one embodiment, the present invention contemplates an isolated nucleic acid encoding an ST2 protein having an amino acid sequence including, but not limited to, SEQ ID NO:1, SEQ ID NO: 3, or SEQ ID NO: 5.

In one embodiment, the present invention contemplates isolated nucleic acids having a sequence including, but not limited to SEQ ID NO: 2, SEQ ID NO: 4, or SEQ ID NO: 6. Primers to ST2 nucleic acid sequences may be constructed for use in a method of reverse transcriptase-polymerase chain reaction (RT-PCR) by generating complementary sequences to any portion of nucleic acid sequences including, but not limited to, SEQ ID NO:2, SEQ ID NO: 4, or SEQ ID NO: 6. In one embodiment, a primer may be approximately 18 bases in length. The entire 18 nucleotide primers can be used, as well as any portion of these sequences of at least fifteen contiguous bases in length. In addition, other oligonucleotides that are fifteen to twenty three nucleotides in length, and that overlap by at least 15 nucleotides, can also be used as primers. In general, additional nucleotides beyond 18 nucleotides for sense primers should generally correspond to nucleotide sequences located on either side of the primer sequence found in the sST2 gene. Additional nucleotides beyond the 18 nucleotides for antisense primers should generally correspond to nucleotide sequences complementary to the sequence recognized by the primer in the sST2 gene.

In one embodiment, the present invention contemplates a method of detecting an infection caused by a dengue virus in a biological sample. The method comprises incubating RNA extracted from the sample with reverse transcriptase and a first sST2 primer of, e.g., 15 to 28 nucleotides, and including at least 15 consecutive nucleotides of, e.g., SEQ ID NO: 2, SEQ ID NO: 4, or SEQ ID NO: 6, wherein the sST2 primer is fully complementary to a region in the dengue viral coding region, for a time and under conditions sufficient to allow double stranded nucleic acid to form; adding a second sST2 primer of, e.g., 15 to 28 nucleotides, wherein the second sST2 primer is identical to a region of at least 15 nucleotides in the dengue viral nucleic acid coding region, and a thermostable DNA polymerase; incubating for a time and under conditions sufficient to allow said double stranded nucleic acid, if any, to be amplified by polymerase chain reaction to form reaction products; and detecting the reaction products as an indication of the presence of dengue virus in the sample.

In one embodiment, the present invention contemplates a method of quantitating dengue virus in a sample. The method includes the steps of mixing RNA extracted from the sample with a known quantity of competitor RNA; incubating the mixture with reverse transcriptase and a first sST2 primer of, e.g., 15 to 28 nucleotides and including at least 15 consecutive nucleotides of, e.g., SEQ ID NO:2, wherein the first sST2 primer is fully complementary to a region in the sST2 nucleic acid complementary to SEQ ID NO:2, for a time and under conditions sufficient to allow double stranded nucleic acid to form; adding a second sST2 primer of, e.g., 15 to 28 nucleotides and including at least 15 consecutive nucleotides of, e.g., SEQ ID NO:6, wherein the second sST2 primer is identical to a region in the sST2 nucleic acid that includes SEQ ID NO:6, and a thermostable DNA polymerase; incubating for a time and under conditions sufficient to allow said double stranded nucleic acid to be amplified by polymerase chain reaction to form reaction products; detecting the reaction products; and comparing the amount of the reaction product obtained with the amount obtained in the absence of said competitor RNA.

Another method of quantitating dengue virus has the following steps: mixing RNA extracted from the sample with a known quantity of competitor RNA; incubating the mixture with reverse transcriptase and a first sST2 primer of, e.g., 15 to 28 nucleotides and including at least 15 consecutive nucleotides of, e.g., SEQ ID NO:2, wherein the first sST2 primer is fully complementary to a region in the sST2 nucleic acid complementary to SEQ ID NO:2, for a time and under conditions sufficient to allow double stranded nucleic acid to form; adding a second sST2 primer of, e.g., 15 to 28 nucleotides and including at least 15 consecutive nucleotides of, e.g., SEQ ID NO:6, wherein the second sST2 primer is identical to a region in the sST2 nucleic acid that includes SEQ ID NO:6, and a thermostable DNA polymerase; incubating for a time and under conditions sufficient to allow said double stranded nucleic acid to be amplified by polymerase chain reaction to form reaction products; detecting the reaction products; and quantitating the reaction products obtained, by comparison to known amounts of competitor RNA.

V. Administration of Soluble Interleukin-1 Like Receptor 1 (sST2) Polypeptides

In one embodiment, the present invention provides therapeutic compositions comprising an effective amount of an sST2 formulation and a suitable diluent and carrier. In one embodiment, the present invention contemplates a method for enhancing sST2 responses comprising administering an effective amount of a small molecule (i.e., for example, a drug or other low molecular weight organic compound).

For therapeutic uses, a purified sST2 formulation is administered to a patient for treatment in a manner appropriate to the indication (i.e., for example, a dengue fever condition) Thus, for example, an sST2 formulation may be administered to a subject for enhancing sST2 protein expression. In one embodiment, an sST2 formulation is administered in the form of a composition comprising purified protein (i.e., for example, an sST2 polypeptide) in conjunction with physiologically acceptable carriers, excipients or diluents. Neutral buffered saline or saline mixed with serum albumin are exemplary appropriate diluents. Preferably, product is lyophilized using appropriate excipient solutions (e.g., sucrose) as diluents. Appropriate dosages can be determined in trials; generally, sST2 formulations comprise dosages of from about 1 ng/kg/day to about 10 mg/kg/day, and more preferably from about 500 μg/kg/day to about 5 mg/kg/day, are expected to induce a biological effect. sST2 formulations can be administered, for example, for the purpose of enhancing sST2 synthesis, release, and/or activity in a patient (i.e., for example, a human patient).

The present invention further provides pharmaceutical compositions (e.g., comprising sST2 mRNA and/or sST2 polypeptides, as described above). The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product.

Agents that enhance uptake of oligonucleotides at the cellular level may also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Certain embodiments of the invention provide pharmaceutical compositions containing (a) one or more mRNA compounds and/or (b) one or more other antiviral agents. Examples of such antiviral agents include, but are not limited to, AZT (Glaxo Wellcome), 3TC (Glaxo Wellcome), ddI (Bristol-Myers Squibb), ddC (Hoffmann-La Roche), D4T (Bristol-Myers Squibb), abacavir (Glaxo Wellcome), nevirapine (Boehringher Ingelheim), delavirdine (Pharmaciand Upjohn), efavirenz (DuPont Pharmaceuticals), saquinavir (Hoffmann-La Roche), ritonavir (Abbott Laboratories), indinavir (Merck and Company), nelfinavir (Agouron Pharmaceuticals), amprenavir (Glaxo Wellcome), adefovir (Gilead Sciences), hydroxyurea (Bristol-Meyers Squibb), AL-721 (lipid mixture) manufactured by Ethigen Corporation and Matrix Research Laboratories; Amphotericin B methyl ester; Ampligen (mismatched RNA) developed by DuPont/HEM Research; anti-AIDS antibody (Nisshon Food); 1 AS-101 (heavy metal based immunostimulant); Betaseron (β-interferon) manufactured by Triton Biosciences (Shell Oil); butylated hydroxytoluene; Carrosyn (polymannoacetate); Castanospermine; Contracan (stearic acid derivative); Creme Pharmatex (containing benzalkonium chloride) manufactured by Pharmalec; CS-87 (5-unsubstituted derivative of Zidovudine), Cytovene (ganciclovir) manufactured by Syntex Corporation; dextran sulfate; D-penicillamine (3-mercapto-D-valine) manufactured by Carter-Wallace and Degussa Pharmaceutical; Foscarnet (trisodium phosphonoformate) manufactured by Astra AB; fusidic acid manufactured by Leo Lovens; glycyrrhizin (a constituent of licorice root); HPA-23 (ammonium-21-tungsto-9-antimonate) manufactured by Rhone-Poulenc Sante; human immune virus antiviral developed by Porton Products International; Ornidyl (eflornithine) manufactured by Merrell-Dow; nonoxinol; pentamidine isethionate (PENTAM-300) manufactured by Lypho Med; Peptide T (octapeptide sequence) manufactured by Peninsula Laboratories; Phenyloin (Warner-Lambert); Ribavirin; Rifabutin (ansamycin) manufactured by Adria Laboratories; CD4-IgG2 (Progenies Pharmaceuticals) or other CD4-containing or CD4-based molecules; T-20 (Trimeris); Trimetrexate manufactured by Warner-Lambert Company; SK-818 (germanium-derived antiviral) manufactured by Sanwa Kagaku; suramin and analogues thereof manufactured by Miles Pharmaceuticals; UA001 manufactured by Ueno Fine Chemicals Industry; and alpha-interferon, manufactured by Glaxo Wellcome.

Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. Two or more combined compounds may be used together or sequentially.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual oligonucleotides, and can generally be estimated based on EC₅₀ₛ found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the oligonucleotide is administered in maintenance doses, ranging from 0.01 μg to 100 g per kg of body weight, once or more daily, to once every 20 years.

### VI. Flavivirus Infections

Flaviriuses are a genus of the family Flaviviridae of single-stranded RNA viruses that are transmitted by arthropod vectors and especially by ticks and mosquitoes. Numerous diseases a caused by such viruses including, but not limited to, dengue fever, Japanese B encephalitis, Saint Louis encephalitis, West Nile fever, West Nile encephalitis, West Nile meningitis Hantaan fever, Sin Nombre fever, and yellow fever. Some of these diseases have mild symptoms (i.e., for example, dengue fever), others are fatal (i.e., for example, West Nile encephalitis).

A. Dengue Fever (DF)

Dengue virus infection is an acute infection cleared approximately within one week (22). Dengue fever is a virus-based disease spread by mosquitoes. DF is caused by four different arboviruses (i.e., for example, Flaiviridae). DF spread by the bite of mosquitoes, most commonly the mosquito Aedes aegypti, which found in tropic and subtropic regions (i.e., for example, Southeast Asia, Indonesian archipelago into northeastern Australia, Sub-Saharan Africa, or South and Central America). Dengue fever, therefore, is also common among world travelers.

DF generally lasts a week or more, is uncomfortable, but not deadly and a full recovery is usually expected. DF should not be confused with Dengue hemorrhagic fever, which is a separate disease and frequently deadly.

Dengue fever begins with a sudden high fever, often to 104-105 degrees Fahrenheit. A flat, red rash may appear over most of the body early during the fever. A second rash, measles-like in appearance, appears later in the disease. Infected people may have increased skin sensitivity and are very uncomfortable. Other symptoms of dengue fever include, but are not limited to, headache, joint aches, muscle aches, nausea, swollen lymph nodes, and/or vomiting.

Diagnostic testing that may be performed to diagnose this condition include, but are not limited to, complete blood count (CBC), serology studies to look for antibodies to flaviviruses, and/or antibody titer for flavivirus types (i.e., for example dengue viruses). Until the present invention, there was no specific treatment for dengue fever. Fluids are necessary if there are signs of dehydration. Acetaminophen (i.e., for example, Tylenol®) is used to treat a high fever but aspirin should be avoided.

B. West Nile Fever

West Nile virus was first identified in 1937 in Uganda in eastern Africa. It was first identified in the United States in the summer of 1999 in New York. Since then, the virus has spread throughout the United States. The West Nile virus is a type of organism called a flavivirus. Although it is not necessary to understand the mechanism of an invention, it is believed that West Nile virus is spread when a mosquito bites an infected bird and then bites a person. Mosquitoes carry the highest amounts of West Nile virus in the early fall, which is why the rate of the disease increases in late August to early September. The risk of disease decreases as the weather becomes colder and mosquitoes die off.

Mild, flu-like illness is often called West Nile fever. More severe forms of disease, which can be life threatening, may be called West Nile encephalitis or West Nile meningitis. Risk factors for developing a more severe form of West Nile virus include, but are not limited to, conditions that weaken the immune system, such as HIV, organ transplants, and recent chemotherapy; pregnancy; or advanced age.

West Nile virus may also be spread through blood transfusions and organ transplantation. It is possible for an infected mother to spread the virus to her child through breast milk. The mildest West Nile disease, is generally called West Nile fever, has some or all of the following symptoms: fever, headache, back pain, muscle aches, lack of appetite, sore throat, nausea, vomiting, abdominal pain, and diarrhea. These symptoms usually last for 3 to 6 days. The more severe West Nile diseases (i.e., for example, encephalitis and/or meningitis), may also have symptoms including, but not limited to: muscle weakness, stiff neck, confusion or change in clarity of thinking, or loss of consciousness. Further, a rash may be present in 20-50% of patients and true muscle weakness in the presence of other related symptoms is suggestive of a West Nile virus infection.

Tests to diagnose West Nile virus may include, but are not limited to, complete blood count, lumbar puncture and cerebrospinal fluid (CSF) testing, or head computer tomography (CT) and multiple resonance intensity (MRI) scanning. However, a definitive diagnosis may be obtained using a serology test, which checks a blood or CSF sample for antibodies against the virus. Alternatively, the virus can also be identified in body fluids using polymerase chain reaction (PCR).

Antiviral drug treatments (i.e., for example, ribavirin) are still in the research phase, therefore standard care (i.e., bedrest, and/or fluids) are recommended to prevent secondary complications. Complications from mild West Nile virus infection are extremely rare. In contrast, complications from severe West Nile virus infection include permanent brain damage or muscle weakness (sometimes similar to polio), and death.

In general, the likely outcome of a mild West Nile virus infection is excellent. For patients with severe cases of West Nile virus infection, the outlook is more guarded. West Nile encephalitis or meningitis has the potential to lead to brain damage and death. Approximately 10% of patients with brain inflammation do not survive.

C. Yellow Fever

Yellow fever is a viral infection transmitted by mosquito bites that causes fever, jaundice, kidney failure, and bleeding. The responsible virus is believed to be a single-stranded RNA virus of the genus Flavivirus (species Yellow fever virus) transmitted especially by the yellow-fever mosquito—called also the yellow jack mosquito. The disease is most common in South America and in sub-Saharan Africa. Yellow fever ranges in severity. Severe infections with internal bleeding and fever (hemorrhagic fever) are deadly in 25-50% of cases.

Anyone can get yellow fever, but the elderly have a higher risk of severe infection. If a person is bitten by an infected mosquito, symptoms usually develop 3 to 6 days later. Yellow fever can be divided into at least three stages: 1) Early stage: Headache, muscle aches, fever, loss of appetite, vomiting, and jaundice are common. After approximately 3 to 4 days, victims often experience brief remission; 2) Period of remission: After a few days (3 to 4) fever and other symptoms go away. Most individuals will recover at this stage, but others may move onto the third, most dangerous stage (intoxication stage) within 24 hours; 3) Period of intoxication: Multi-organ dysfunction occurs. This includes, but is not limited to, liver and kidney failure, bleeding disorders/hemorrhage, brain dysfunction including, but not limited to, delirium, seizures, coma, shock, and death.

In general yellow fever symptoms include, but are not limited to, fever, headache, muscle aches (myalgia), vomiting, red eyes, red face, red tongue, jaundice, bleeding and/or hemorrhage, decreased urination, arrhythmias, heart dysfunction, vomiting blood, delirium, seizures, or coma. A person with advanced yellow fever may also show signs of liver failure, renal failure, and shock. A symptomatic diagnosis may be confirmed by blood tests that reveal the virus, viral antigens, or antibodies.

Currently, there is no specific treatment for yellow fever. Treatment for symptoms may include intravenous fluids, blood products for severe bleeding, and dialysis for renal failure. Further secondary complications may occur, including but not limited to, kidney failure, disseminated intravascular coagulation (DIC), secondary bacterial infections, liver failure, parotitis, shock, coma, or death.

D. Encephalitis

Encephalitis is an inflammation (irritation and swelling) of the brain, usually caused by infections. Encephalitis is most often caused by a viral infection, and many types of viruses may cause it. Exposure to viruses can occur through insect bites, food or drink contamination, inhalation of respiratory droplets from an infected person, or skin contact. In rural areas, arboviruses (i.e., for example, flaviviruses such as Japanese B virus and/or Saint Louis virus)—carried by mosquitoes or ticks, or accidentally ingested, are the most common cause. Encephalitis is relatively uncommon but still affects approximately 1,500 people per year in the U.S. The elderly and infants are more vulnerable and may have a more severe course of the disease.

Once an encephalitis virus has entered the bloodstream, it may localize in the brain, causing inflammation of brain tissue and surrounding membranes. White blood cells invade the brain tissue as they try to fight off the infection. The brain tissue swells (cerebral edema), which may cause destruction of nerve cells, bleeding within the brain (intracerebral hemorrhage), and brain damage.

Encephalitis symptoms may include, but are not limited to, fever, headache, vomiting, light-sensitivity, stiff neck and/or back, confusion, disorientation, drowsiness, clumsiness, unsteady gait, irritability, or poor temper control. More serious symptoms can also develop including, but not limited to, loss of consciousness, poor responsiveness, stupor, coma, seizures, muscle weakness and/or paralysis, memory loss (amnesia), impaired short-term memory or impaired long-term memory. Some behavioral symptoms may also be present including, but not limited to, a “flat” mood or lack of discernible mood, or mood inappropriate for the situation, diminished interest in daily activities, inflexibility, extreme self-centeredness, indecisiveness, withdrawal from social interaction, or impaired judgment

An examination may show signs of meningeal irritation (especially neck stiffness), increased intracranial pressure, or other neurologic symptoms such as muscle weakness, mental confusion, speech problems, and abnormal reflexes. The patient may have a skin rash, mouth ulcers, and signs of involvement of other organs such as the liver and lungs. A lumbar puncture test and cerebrospinal fluid (CSF) examination may show clear fluid, high pressure, high white blood cell count and high protein levels—indications of inflammation. Blood may be present in the CSF.

Sometimes the virus can be detected in CSF, blood, or urine through a laboratory test called viral culture. In some cases, viral PCR (polymerase chain reaction, a test able to detect very tiny amounts of viral DNA) may identify the virus. Serology tests may also provide evidence of viral infection. Alternatively, an electroencephalogram (EEG) may provide indirect clues for the diagnosis of encephalitis. Some EEG wave patterns may suggest a seizure disorder, or point to a specific virus as cause of the infection. Certain EEG wave patterns can suggest encephalitis due to herpes, for instance. A brain MRI, which provides high-quality pictures of the brain, or a CT scan of the head may be used to determine internal bleeding or specific areas of brain inflammation.

Presently, no specific antiviral drugs are available to combat the infection. Consequently, supportive care (i.e., for example, rest, nutrition, and/or fluids) is usually provided to relieve symptoms. Antiviral medications, such as acyclovir (Zovirax) and foscarnet (Foscavir), may be useful but are not clinically effective.

The outcome viral encephalitis infections varies. Some cases are mild, short, and relatively harmless, followed by full recovery. Other cases are severe, and permanent impairment or death is possible. The acute phase normally lasts for 1 to 2 weeks, with gradual or sudden disappearance of fever and neurologic symptoms. Neurologic symptoms may require many months before full recovery.

### VII. Bunyavirus Infections

The virus family Bunyaviridae, are rodent-borne negative-stranded RNA viruses. Members of the genus Hantavirus have been identified as etiologic agents of two severe human diseases: hemorrhagic fever with renal syndrome (HFRS), which is caused by the Old World hantaviruses, and hantavirus pulmonary syndrome (HPS), which is caused by the New World hantaviruses. Case fatality is considerably higher for HPS (up to 40%) than for HFRS (between 0.1 and 15%). The major target in human hantavirus infection is the microvascular endothelium, and severe hantavirus disease in humans has been attributed to microvascular leakage. Several Old and New World hantaviruses have not been associated with any human disease. The basis for disease in humans, and differences between pathogenic and nonpathogenic hantaviruses, remains unclear; however, innate immune responses likely plays a role.

A. Hantaan Fever

Hantaan fever (also known as Hantavirus disease) characterized by symptoms that resemble the flu, followed by respiratory failure. Hantaan fever is a potentially fatal respiratory illness first identified in the United States Southwest. Since that discovery, hantavirus disease has been reported in every western state, and in many eastern states.

Hantavirus is carried by rodents, particularly deer mice, and is present in their urine and feces. The virus does not cause disease in the carrier animal. Humans are thought to become infected when they are exposed to contaminated dust from the nests or droppings of mice.

The disease is not, however, passed between humans. Contaminated dust is often encountered when cleaning long-vacated dwellings, sheds, or other enclosed areas. Initial symptoms of hantavirus disease closely resemble the flu. The disease begins abruptly with fever, chills, muscle aches, headache, nausea and vomiting, and malaise. A dry cough may be present. The fever may be higher in younger people than in older people.

For a very short period, the infected person feels somewhat better, but this is followed within a day or two by an increased respiratory rate caused by a seepage of fluid into the lungs. The initial shortness of breath is subtle and the patient may be unaware of it, but progression is rapid. The patient ultimately develops respiratory failure.

An effective treatment for hantavirus is not yet available. Even with intensive therapy, more than half of the diagnosed cases have been fatal. Hantaan virus symptoms may include, but are not limited to, chills, dry cough, fever, general ill feeling (malaise), headache, muscle aches, rapid shallow breathing, respiratory failure, or shortness of breath. Other indications of hantaan virus infection may include, but are not limited to, hypoxia, hypotension, or acute respiratory distress syndrome.

Diagnostic tests for Hantaan fever include, but are not limited to, complete blood count (i.e., for example, elevated white blood cells); platelet count (i.e., for example, <150,000 and decreasing), chest X-ray (i.e., for example, lung tissue invasion/infiltration), liver enzymes (i.e., for example, LDH elevation), decreased serum albumin, increased hematocrit, serological testing for hantavirus presence.

Because the breathing problems progress rapidly, cardiorespiratory failure is common associated with a high death rate (i.e., for example over 50%). Oxygen therapy is used with respiratory support from a breathing tube (i.e., for example, an endotracheal tube) and/or ventilator.

B. Sin Nombre Fever

Sin Nombre virus causes the majority of Hantavirus pulmonary syndrome (HPS cases) in the United States, and the deer mouse (Peromyscus maniculatus) is its predominant reservoir. HPS is a rodent-borne viral disease characterized by severe pulmonary illness and a case-fatality ratio of 30%-40%.

HPS is characterized by a febrile illness (i.e., temperature >101.0° F.) associated with bilateral diffuse interstitial edema of the lungs developing within 72 hours of hospitalization in a previously healthy person; radiographically, the edema can resemble acute respiratory distress syndrome (1). Annually, the majority of HPS cases occur in spring and summer; however, the seasonality of HPS can vary by elevation, location, and biome, and cases have been identified throughout the winter and early spring. Since recognition of the disease in 1993, CDC has confirmed 438 cases of HPS reported from 30 states among residents of 32 states; 35% (154) of these cases were fatal.

HPS typically begins as headache, fever, and myalgia and is soon followed by pulmonary edema, which often leads to severe respiratory compromise; thrombocytopenia, presence of immunoblasts, and hemoconcentration are characteristic laboratory findings (1). Other than supportive care, no treatment exists for hantavirus infection.

### VIII. Expression Platforms

The present invention provides recombinant expression vectors for expression of an sST2 polypeptide, and host cells transformed with the expression vectors. Any suitable expression system may be employed. The vectors include a DNA encoding an sST2 polypeptide, operably linked to suitable transcriptional or translational regulatory nucleotide sequences, such as those derived from a mammalian, microbial, viral, or insect gene. Examples of regulatory sequences include transcriptional promoters, operators, or enhancers, an mRNA ribosomal binding site, and appropriate sequences which control transcription and translation initiation and termination. Nucleotide sequences are operably linked when the regulatory sequence functionally relates to an sST2 DNA sequence. Thus, a promoter nucleotide sequence is operably linked to an sST2 DNA sequence if the promoter nucleotide sequence controls the transcription of an sST2 DNA sequence. An origin of replication that confers the ability to replicate in the desired host cells, and a selection gene by which transformants are identified, are generally incorporated into the expression vector.

In addition, a sequence encoding an appropriate signal peptide can be incorporated into expression vectors. A DNA sequence for a signal peptide (secretory leader) may be fused in frame to an sST2 nucleic acid sequence so that an sST2 peptide is initially translated as a fusion protein comprising the signal peptide. A signal peptide that is functional in the intended host cells promotes extracellular secretion of an sST2 antagonist polypeptide. The signal peptide is cleaved from an sST2 polypeptide upon secretion from the cell.

Suitable host cells for expression of an sST2 polypeptide include prokaryotes, yeast or higher eukaryotic cells. Appropriate cloning and expression vectors for use with bacterial, fungal, yeast, and mammalian cellular hosts are described, for example, in Pouwels et al. Cloning Vectors: A Laboratory Manual, Elsevier, N.Y., (1985). Cell-free translation systems could also be employed to produce an sST2 polypeptide using RNAs derived from DNA constructs disclosed herein.

Prokaryotes include gram negative or gram positive organisms, for example, E. coli or Bacilli. Suitable prokaryotic host cells for transformation include, for example, E. coli, Bacillus subtilis, Salmonella typhimurium, and various other species within the genera Pseudomonas, Streptomyces, and Staphylococcus. In a prokaryotic host cell, such as E. coli, an sST2 antagonist polypeptide may include an N-terminal methionine residue to facilitate expression of the recombinant polypeptide in the prokaryotic host cell. The N-terminal Met may be cleaved from the expressed recombinant sST2 polypeptide.

Expression vectors for use in prokaryotic host cells generally comprise one or more phenotypic selectable marker genes. A phenotypic selectable marker gene is, for example, a gene encoding a protein that confers antibiotic resistance or that supplies an autotrophic requirement. Examples of useful expression vectors for prokaryotic host cells include those derived from commercially available plasmids such as the cloning vector pBR322 (ATCC 37017). pBR322 contains genes for ampicillin and tetracycline resistance and thus provides simple means for identifying transformed cells. An appropriate promoter and an sST2 DNA sequence are inserted into the pBR322 vector. Other commercially available vectors include, for example, pKK223-3 (Pharmacia Fine Chemicals, Uppsala, Sweden) and pGEM1 (Promega Biotec, Madison, Wis., USA).

Promoter sequences commonly used for recombinant prokaryotic host cell expression vectors include p-lactamase (penicillinase), lactose promoter system (Chang et al., Nature 275:615, 1978; and Goeddel et al., Nature 281:544, 1979), tryptophan (trp) promoter system (Goeddel et al., Nucl. Acids Res. 8:4057, 1980; and EP-A-36776) and tac promoter (Maniatis, Molecular Cloning: A Laboratory Manual, Cold Spring Harbor Laboratory, p. 412, 1982). A particularly useful prokaryotic host cell expression system employs a phage λ P_{L} promoter and a cI857ts thermolabile repressor sequence. Plasmid vectors available from the American Type Culture Collection which incorporate derivatives of the λ P_{L} promoter include plasmid pHUB2 (resident in E. coli strain JMB9, ATCC 37092) and pPLc28 (resident in E. coli RR1, ATCC 53082).

An sST2 polypeptide alternatively may be expressed in yeast host cells, preferably from the Saccharomyces genus (e.g., S. cerevisiae). Other genera of yeast, such as Pichia or Kluyveromyces, may also be employed. Yeast vectors will often contain an origin of replication sequence from a yeast plasmid, an autonomously replicating sequence (ARS), a promoter region, sequences for polyadenylation, sequences for transcription termination, and a selectable marker gene. Suitable promoter sequences for yeast vectors include, among others, promoters for metallothionein, 3-phosphoglycerate kinase (Hitzeman et al., J. Biol. Chem. 255:2073, 1980) or other glycolytic enzymes (Hess et al., J. Adv. Enzyme Reg. 7:149, 1968; and Holland et al., Biochem. 17:4900, 1978), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. Other suitable vectors and promoters for use in yeast expression are further described in Hitzeman, EPA-73,657. Another alternative is the glucose-repressible ADH2 promoter described by Russell et al. (J. Biol. Chem. 258:2674, 1982) and Beier et al. (Nature 300:724, 1982). Shuttle vectors replicable in both yeast and E. coli may be constructed by inserting DNA sequences from pBR322 for selection and replication in E. coli (Amp^{r} gene and origin of replication) into the above-described yeast vectors.

The yeast α-factor leader sequence may be employed to direct secretion of an sST2 polypeptide. The α-factor leader sequence is often inserted between the promoter sequence and the structural gene sequence. See, e.g., Kurjan et al., Cell 30:933, 1982 and Bitter et al., Proc. Natl. Acad. Sci. USA 81:5330, 1984. Other leader sequences suitable for facilitating secretion of recombinant polypeptides from yeast hosts may also be used. Further, a leader sequence may be modified near its 3′ end to contain one or more restriction sites. This will facilitate fusion of the leader sequence to the structural gene.

Yeast transformation protocols are described. Hinnen et al., Proc. Natl. Acad. Sci. USA 75:1929, 1978. The Hinnen et al. protocol selects for Trp⁺ transformants in a selective medium, wherein the selective medium consists of 0.67% yeast nitrogen base, 0.5% amino acids, 2% glucose, 10 μg/ml adenine and 20 μg/ml uracil.

Yeast host cells transformed by vectors containing an ADH2 promoter sequence may be grown for inducing expression in a “rich” medium. An example of a rich medium is one consisting of 1% yeast extract, 2% peptone, and 1% glucose supplemented with 80 μg/ml adenine and 80 μm/ml uracil. Derepression of the ADH2 promoter occurs when glucose is exhausted from the medium.

Mammalian or insect host cell culture systems could also be employed to express recombinant sST2 polypeptides. Bacculovirus systems for production of heterologous proteins in insect cells are reviewed by Luckow and Summers, Bio/Technology 6:47 (1988). Established cell lines of mammalian origin also may be employed. Examples of suitable mammalian host cell lines include the COS-7 line of monkey kidney cells (ATCC CRL 1651) (Gluzman et al., Cell 23:175, 1981), L cells, C127 cells, 3T3 cells (ATCC CCL 163), Chinese hamster ovary (CHO) cells, HeLa cells, and BHK (ATCC CRL 10) cell lines, and the CVI/EBNA cell line derived from the African green monkey kidney cell line CVI (ATCC CCL 70) as described by McMahan et al. (EMBO J. 10: 2821, 1991).

Transcriptional and translational control sequences for mammalian host cell expression vectors may be excised from viral genomes. Commonly used promoter sequences and enhancer sequences are derived from Polyoma virus, Adenovirus 2, Simian Virus 40 (SV40), and human cytomegalovirus. DNA sequences derived from the SV40 viral genome, for example, SV40 origin, early and late promoter, enhancer, splice, and polyadenylation sites may be used to provide other genetic elements for expression of a structural gene sequence in a mammalian host cell. Viral early and late promoters are particularly useful because both are easily obtained from a viral genome as a fragment which may also contain a viral origin of replication (Fiers et al., Nature 273:113, 1978). Smaller or larger SV40 fragments may also be used, provided the approximately 250 bp sequence extending from the Hind III site toward the BglI site located in the SV40 viral origin of replication site is included.

Expression vectors for use in mammalian host cells can be constructed as disclosed by Okayama and Berg (Mol. Cell. Biol. 3:280, 1983), for example. A useful system for stable high level expression of mammalian cDNAs in C127 murine mammary epithelial cells can be constructed substantially as described by Cosman et al. (Mol. Immunol. 23:935, 1986). A high expression vector, PMLSV N1/N4, described by Cosman et al., Nature 312:768, 1984 has been deposited as ATCC 39890. Additional mammalian expression vectors are described in EP-A-0367566, and in WO 91/18982. As one alternative, the vector may be derived from a retrovirus. Additional suitable expression systems are described in the examples below.

One preferred expression system employs Chinese hamster ovary (CHO) cells and an expression vector designated PG5.7. This expression vector is described in U.S. patent application Ser. No. 08/586,509, filed Jan. 11, 1996, which is hereby incorporated by reference. PG5.7 components include a fragment of CHO cell genomic DNA, followed by a CMV-derived promoter, which is followed by a sequence encoding an adenovirus tripartite leader, which in turn is followed by a sequence encoding dihydrofolate reductase (DHFR). These components were inserted into the plasmid vector pGEM1 (Promega, Madison, Wis.). DNA encoding an sST2 polypeptide (or fusion protein containing an sST2 peptide) may be inserted between the sequences encoding the tripartite leader and DHFR. Methotrexate may be added to the culture medium to increase expression levels.

The fragment of CHO cell genomic DNA in vector PG5.7 enhances expression of an sST2 protein. A phage lysate containing a fragment of genomic DNA isolated from CHO cells was deposited with the American Type Culture Collection on Jan. 4, 1996, and assigned accession number ATCC 97411. Vector PG5.7 contains nucleotides 8671 through 14507 of the CHO genomic DNA insert in strain deposit ATCC 97411.

For expression of an sST2 polypeptide, a type II protein lacking a native signal sequence, a heterologous signal sequence or leader functional in mammalian host cells may be added. Examples include the signal sequence for interleukin-7 (IL-7) described in U.S. Pat. No. 4,965,195, the signal sequence for interleukin-2 receptor described in Cosman et al., Nature 312:768 (1984); the interleukin-4 receptor signal peptide described in EP 367,566; the type I interleukin-1 receptor signal peptide described in U.S. Pat. No. 4,968,607; and the type I interleukin-1 receptor signal peptide described in EP 460,846.

### IX. Protein Purification

The present invention provides purified sST2 proteins, which may be produced by recombinant expression systems as described above or purified from naturally occurring cells. The desired degree of purity may depend on the intended use of the protein. A relatively high degree of purity is desired when the protein is to be administered in vivo, for example. Advantageously, sST2 polypeptides are purified such that no protein bands corresponding to other proteins are detectable by SDS-polyacrylamide gel electrophoresis (SDS-PAGE). As demonstrated herein, multiple bands corresponding to sST2 protein may be detected by SDS-PAGE, due to differential glycosylation, variations in post-translational processing. In one embodiment, an SDS-PAGE detection provides purified sST2 protein when no visual bands corresponding to different (non-sST2) proteins are visualized. sST2 most preferably is purified to substantial homogeneity, as indicated by a single protein band upon analysis by SDS-PAGE. The protein band may be visualized by silver staining, Coomassie blue staining, or (if the protein is radiolabeled) by autoradiography.

One process for producing the sST2 protein comprises culturing a host cell transformed with an expression vector comprising a DNA sequence that encodes sST2 under conditions such that sST2 is expressed. The sST2 protein is then recovered from the culture (from the culture medium or cell extracts). Procedures for purifying the recombinant sST2 will vary according to such factors as the type of host cells employed and whether or not the sST2 is secreted into the culture medium.

For example, when expression systems that secrete the recombinant protein are employed, the culture medium first may be concentrated using a commercially available protein concentration filter, for example, an Amicon or Millipore Pellicon ultrafiltration unit. Following the concentration step, the concentrate can be applied to a purification matrix such as a gel filtration medium. Alternatively, an anion exchange resin can be employed, for example, a matrix or substrate having pendant diethylaminoethyl (DEAE) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. Sulfopropyl groups are preferred. Finally, one or more reversed-phase high performance liquid chromatography (RP-HPLC) steps employing hydrophobic RP-HPLC media, (e.g., silica gel having pendant methyl or other aliphatic groups) can be employed to further purify sST2. Some or all of the foregoing purification steps, in various combinations, can be employed to provide a purified sST2 protein.

Recombinant protein produced in bacterial culture may be isolated by initial disruption of the host cells, centrifugation, extraction from cell pellets if an insoluble polypeptide, or from the supernatant fluid if a soluble polypeptide, followed by one or more concentration, salting-out, ion exchange, affinity purification or size exclusion chromatography steps. Finally, RP-HPLC can be employed for final purification steps. Microbial cells can be disrupted by any convenient method, including freeze-thaw cycling, sonication, mechanical disruption, or use of cell lysing agents.

Transformed yeast host cells are preferably employed to express sST2 as a secreted polypeptide. This simplifies purification. Secreted recombinant polypeptide from a yeast host cell fermentation can be purified by methods analogous to those disclosed by Urdal et al. (J. Chromatog. 296:171, 1984). Urdal et al. describe two sequential, reversed-phase HPLC steps for purification of recombinant human IL-2 on a preparative HPLC column.

Alternatively, sST2 polypeptides can be purified by immunoaffinity chromatography. An affinity column containing an antibody that binds sST2 may be prepared by conventional procedures and employed in purifying sST2.

### X. Fusion Proteins

The present invention also provides fusion proteins incorporating all or part of a sST2 protein. Accordingly, in some embodiments of the present invention, the coding sequences for the polypeptides can be incorporated as a part of a fusion gene including a nucleotide sequence encoding a different polypeptide.

sST2 polypeptide fusions can comprise peptides added to facilitate purification and identification of sST2. Such peptides include, for example, poly-His or the antigenic identification peptides described in U.S. Pat. No. 5,011,912 and in Hopp et al., Bio/Technology 6:1204, 1988. One such peptide is the FLAG® peptide, Asp-Tyr-Lys-Asp-Asp-Asp-Asp-Lys (DYKDDDDK) (SEQ ID NO:7), which is highly antigenic and provides an epitope reversibly bound by a specific monoclonal antibody, thus enabling rapid assay and facile purification of expressed recombinant protein. This sequence is also specifically cleaved by bovine mucosal enterokinase at the residue immediately following the Asp-Lys pairing. Fusion proteins capped with this peptide may also be resistant to intracellular degradation in E. coli.

Various techniques for making fusion genes have been reported. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment of the present invention, the fusion gene can be synthesized by conventional techniques including automated DNA synthesizers. Alternatively, in other embodiments of the present invention, PCR amplification of gene fragments can be carried out using anchor primers which give rise to complementary overhangs between two consecutive gene fragments which can subsequently be annealed to generate a chimeric gene sequence (See e.g., Current Protocols in Molecular Biology, supra).

The above-described sST2 proteins that can be used in the present invention, may be produced as fusion proteins, constituting a functional variant of one of the previously described proteins or a functional variant only after the fusion moiety has been eliminated. These fusion proteins include, in particular, fusion proteins that have a content of about 1-300 foreign amino acids, preferably about 1-200 foreign amino acids, particularly preferably about 1-150 foreign amino acids, more preferably about 1-100 foreign amino acids, and most preferably about 1-50 foreign amino acids. Such foreign amino acid sequences may be prokaryotic peptide sequences that can be derived, for example, from E. Coli β-galactosidase.

Other examples of peptide sequences for fusion proteins are peptides that facilitate detection of the fusion protein; they include, but are not limited to, green fluorescent protein or variants thereof. It is also possible to add on at least one “affinity tag” or “protein tag” for the purpose of purifying the previously described proteins. For example, suitable affinity tags enable the fusion protein to be absorbed with high specificity and selectivity to a matrix. This attachment step is then followed by stringent washing with suitable buffers without eluting the fusion protein to any significant extent, and specific elution of the absorbed fusion protein. Examples of the protein tags include, but are not limited to, a (His)₆ tag, a Myc tag, a FLAG tag, a hemagglutinin tag, a glutathione-S-transferase (GST) tag, a tag consisting of an intein flanked by an affinity chitin-binding domain, and a maltose-binding protein (MBP) tag. These protein tags can be located N-terminally, C-terminally and/or internally.

The proteins that can be used in the methods and compositions of the present invention can also be prepared synthetically. Thus, the entire polypeptide, or parts thereof, can, for example, be produced by classical synthesis techniques (e.g., Merrifield technique). Particular preference is given to using polypeptides which have been prepared recombinantly using one of the previously described nucleic acids. Furthermore, proteins of the present invention can be isolated from an organism or from tissue or cells for use in accordance with the present invention. Thus, it is possible, for example, to purify proteins, which can be used in the present invention, from human serum. Abdullah et al., Arch. Biochem. Biophys., 225:306 312 (1983). Furthermore, it is possible to prepare cell lines expressing the proteins of the present invention. These cell lines can then be used for isolating the proteins of interest. Suitable systems for production of recombinant proteins include but are not limited to prokaryotic (e.g., Escherichia coli), yeast (e.g., Saccaromyces cerevisiae), insect (e.g., baculovirus), mammalian (e.g., Chinese hamster ovary), plant (e.g., safflower), and cell-free systems (e.g., rabbit reticulocyte).

### XI. Detection Methodologies

A. Detection of RNA

In some embodiments, detection of a virus infection comprises measuring the expression of corresponding mRNA in a biological sample (i.e., for example, a blood sample, a serum sample, a plasma sample, or a tissue biopsy sample). mRNA expression may be measured by any suitable method, including but not limited to, those disclosed below.

In some embodiments, RNA is detection by Northern blot analysis. Northern blot analysis involves the separation of RNA and hybridization of a complementary labeled probe.

In other embodiments, RNA expression is detected by enzymatic cleavage of specific structures (INVADER assay, Third Wave Technologies; See e.g., U.S. Pat. Nos. 5,846,717, 6,090,543; 6,001,567; 5,985,557; and 5,994,069; each of which is herein incorporated by reference). The INVADER assay detects specific nucleic acid (e.g., RNA) sequences by using structure-specific enzymes to cleave a complex formed by the hybridization of overlapping oligonucleotide probes.

In still further embodiments, RNA (or corresponding cDNA) is detected by hybridization to a oligonucleotide probe. A variety of hybridization assays using a variety of technologies for hybridization and detection are available. For example, in some embodiments, TaqMan assay (PE Biosystems, Foster City, Calif.; See e.g., U.S. Pat. Nos. 5,962,233 and 5,538,848, each of which is herein incorporated by reference) is utilized. The assay is performed during a PCR reaction. The TaqMan assay exploits the 5′-3′ exonuclease activity of the AMPLITAQ GOLD DNA polymerase. A probe consisting of an oligonucleotide with a 5′-reporter dye (e.g., a fluorescent dye) and a 3′-quencher dye is included in the PCR reaction. During PCR, if the probe is bound to its target, the 5′-3′ nucleolytic activity of the AMPLITAQ GOLD polymerase cleaves the probe between the reporter and the quencher dye. The separation of the reporter dye from the quencher dye results in an increase of fluorescence. The signal accumulates with each cycle of PCR and can be monitored with a fluorimeter.

In yet other embodiments, reverse-transcriptase PCR(RT-PCR) is used to detect the expression of RNA. In RT-PCR, RNA is enzymatically converted to complementary DNA or “cDNA” using a reverse transcriptase enzyme. The cDNA is then used as a template for a PCR reaction. PCR products can be detected by any suitable method, including but not limited to, gel electrophoresis and staining with a DNA specific stain or hybridization to a labeled probe. In some embodiments, the quantitative reverse transcriptase PCR with standardized mixtures of competitive templates method described in U.S. Pat. Nos. 5,639,606, 5,643,765, and 5,876,978 (each of which is herein incorporated by reference) is utilized.

B. Detection of Protein

In other embodiments, gene expression in virus infected tissues may be detected by measuring the expression of a protein or polypeptide. Protein expression may be detected by any suitable method. In some embodiments, proteins are detected by immunohistochemistry. In other embodiments, proteins are detected by their binding to an antibody raised against the protein. The generation of antibodies is described below.

Antibody binding may be detected by many different techniques including, but not limited to, (e.g., radioimmunoassay, ELISA (enzyme-linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays, etc.

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled.

In some embodiments, an automated detection assay is utilized. Methods for the automation of immunoassays include those described in U.S. Pat. Nos. 5,885,530, 4,981,785, 6,159,750, and 5,358,691, each of which is herein incorporated by reference. In some embodiments, the analysis and presentation of results is also automated. For example, in some embodiments, software that generates a prognosis based on the presence or absence of a series of proteins corresponding to virus induced markers is utilized.

In other embodiments, the immunoassay described in U.S. Pat. Nos. 5,599,677 and 5,672,480; each of which is herein incorporated by reference.

C. Remote Detection Systems

In some embodiments, a computer-based analysis program is used to translate the raw data generated by the detection assay (e.g., the presence, absence, or amount of a given marker or markers) into data of predictive value for a clinician. The clinician can access the predictive data using any suitable means. Thus, in some preferred embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, wherein the information is provided to medical personal and/or subjects. For example, in some embodiments of the present invention, a sample (e.g., a biopsy or a serum or urine sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (i.e., expression data), specific for the diagnostic or prognostic information desired for the subject.

The profile data is then prepared in a format suitable for interpretation by a treating clinician. For example, rather than providing raw expression data, the prepared format may represent a diagnosis or risk assessment (e.g., likelihood of a virus infection) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose further intervention or counseling based on the results. In some embodiments, the data is used for research use. For example, the data may be used to further optimize the inclusion or elimination of markers as useful indicators of a particular condition or stage of disease.

D. Detection Kits

In other embodiments, the present invention provides kits for the detection and characterization of virus infections. In some embodiments, the kits contain antibodies specific for a protein expressed as a result of a virus infection, in addition to detection reagents and buffers. In other embodiments, the kits contain reagents specific for the detection of mRNA or cDNA (e.g., oligonucleotide probes or primers). In preferred embodiments, the kits contain all of the components necessary to perform a detection assay, including all controls, directions for performing assays, and any necessary software for analysis and presentation of results.

### XII. Antibodies

The present invention provides isolated antibodies (i.e., for example, polyclonal or monoclonal). In one embodiment, the present invention provides monoclonal antibodies that specifically bind to an isolated polypeptide comprised of at least five amino acid residues of the gene expression profile proteins described herein (e.g., sST2). These antibodies find use in the detection methods described above.

A murine hybridoma designated 4E11 produces a monoclonal antibody that binds the peptide DYKDDDDK (SEQ ID NO:7) in the presence of certain divalent metal cations (as described in U.S. Pat. No. 5,011,912), and has been deposited with the American Type Culture Collection under Accession No HB 9259. Expression systems useful for producing recombinant proteins fused to the FLAG® peptide, as well as monoclonal antibodies that bind the peptide and are useful in purifying the recombinant proteins, are available from Eastman Kodak Company, Scientific Imaging Systems, New Haven, Conn.

Preparation of Fusion Proteins Comprising Heterologous Polypeptides Fused to Various portions of antibody-derived polypeptides (including the Fc domain) has been described, e.g., by Ashkenazi et al. (PNAS USA 88:10535, 1991); Byrn et al. (Nature 344:667, 1990); and Hollenbaugh and Aruffo (“Construction of Immunoglobulin Fusion Proteins”, in Current Protocols in Immunology, Supplement 4, pages 10.19.1-10.19.11, 1992), hereby incorporated by reference. In one embodiment of the invention, a sST2 fusion protein is created by fusing sST2 to an Fc region polypeptide derived from an antibody. The term “Fc polypeptide” includes native and mutein forms, as well as truncated Fc polypeptides containing the hinge region that promotes dimerization.

A gene fusion encoding the sST2/Fc fusion protein may be inserted into an appropriate expression vector. The sST2/Fc fusion proteins are allowed to assemble much like antibody molecules, whereupon interchain disulfide bonds form between the Fc polypeptides, yielding divalent sST2. In other embodiments, sST2 may be substituted for the variable portion of an antibody heavy or light chain. If fusion proteins are made with both heavy and light chains of an antibody, it is possible to form an sST2 oligomer with as many as four sST2 extracellular regions.

One suitable Fc polypeptide is the native Fc region polypeptide derived from a human IgG1, which is described in PCT application WO 93/10151, hereby incorporated by reference. Another useful Fc polypeptide is the Fc mutein described in U.S. Pat. No. 5,457,035. The amino acid sequence of the mutein is identical to that of the native Fc sequence presented in WO 93/10151, except that amino acid 19 has been changed from Leu to Ala, amino acid 20 has been changed from Leu to Glu, and amino acid 22 has been changed from Gly to Ala. This mutein Fc exhibits reduced affinity for immunoglobulin receptors.

An antibody against a protein of the present invention may be any monoclonal or polyclonal antibody, as long as it can recognize the protein. Antibodies can be produced by using a protein of the present invention as the antigen according to a conventional antibody or antiserum preparation process.

The present invention contemplates the use of both monoclonal and polyclonal antibodies. Any suitable method may be used to generate the antibodies used in the methods and compositions of the present invention, including but not limited to, those disclosed herein. For example, for preparation of a monoclonal antibody, protein, as such, or together with a suitable carrier or diluent is administered to an animal (e.g., a mammal) under conditions that permit the production of antibodies. For enhancing the antibody production capability, complete or incomplete Freund's adjuvant may be administered. Normally, the protein is administered once every 2 weeks to 6 weeks, in total, about 2 times to about 10 times. Animals suitable for use in such methods include, but are not limited to, primates, rabbits, dogs, guinea pigs, mice, rats, sheep, goats, etc.

For preparing monoclonal antibody-producing cells, an individual animal whose antibody titer has been confirmed (e.g., a mouse) is selected, and 2 days to 5 days after the final immunization, its spleen or lymph node is harvested and antibody-producing cells contained therein are fused with myeloma cells to prepare the desired monoclonal antibody producer hybridoma. Measurement of the antibody titer in antiserum can be carried out, for example, by reacting the labeled protein, as described hereinafter and antiserum and then measuring the activity of the labeling agent bound to the antibody. The cell fusion can be carried out according to known methods, for example, the method described by Koehler and Milstein (Nature 256:495 (1975)). As a fusion promoter, for example, polyethylene glycol (PEG) or Sendai virus (HVJ), preferably PEG is used.

Examples of myeloma cells include NS-1, P3U1, SP2/0, AP-1 and the like. The proportion of the number of antibody producer cells (spleen cells) and the number of myeloma cells to be used is preferably about 1:1 to about 20:1. PEG (preferably PEG 1000-PEG 6000) is preferably added in concentration of about 10% to about 80%. Cell fusion can be carried out efficiently by incubating a mixture of both cells at about 20° C. to about 40° C., preferably about 30° C. to about 37° C. for about 1 minute to 10 minutes.

Various methods may be used for screening for a hybridoma producing the antibody (e.g., against a tumor antigen or autoantibody of the present invention). For example, where a supernatant of the hybridoma is added to a solid phase (e.g., microplate) to which antibody is adsorbed directly or together with a carrier and then an anti-immunoglobulin antibody (if mouse cells are used in cell fusion, anti-mouse immunoglobulin antibody is used) or Protein A labeled with a radioactive substance or an enzyme is added to detect the monoclonal antibody against the protein bound to the solid phase. Alternately, a supernatant of the hybridoma is added to a solid phase to which an anti-immunoglobulin antibody or Protein A is adsorbed and then the protein labeled with a radioactive substance or an enzyme is added to detect the monoclonal antibody against the protein bound to the solid phase.

Selection of the monoclonal antibody can be carried out according to any known method or its modification. Normally, a medium for animal cells to which HAT (hypoxanthine, aminopterin, thymidine) are added is employed. Any selection and growth medium can be employed as long as the hybridoma can grow. For example, RPMI 1640 medium containing 1% to 20%, preferably 10% to 20% fetal bovine serum, GIT medium containing 1% to 10% fetal bovine serum, a serum free medium for cultivation of a hybridoma (SFM-101, Nissui Seiyaku) and the like can be used. Normally, the cultivation is carried out at 20° C. to 40° C., preferably 37° C. for about 5 days to 3 weeks, preferably 1 week to 2 weeks under about 5% CO₂ gas. The antibody titer of the supernatant of a hybridoma culture can be measured according to the same manner as described above with respect to the antibody titer of the anti-protein in the antiserum.

Separation and purification of a monoclonal antibody (e.g., against a virus induced marker of the present invention) can be carried out according to the same manner as those of conventional polyclonal antibodies such as separation and purification of immunoglobulins, for example, salting-out, alcoholic precipitation, isoelectric point precipitation, electrophoresis, adsorption and desorption with ion exchangers (e.g., DEAE), ultracentrifugation, gel filtration, or a specific purification method wherein only an antibody is collected with an active adsorbent such as an antigen-binding solid phase, Protein A or Protein G and dissociating the binding to obtain the antibody.

Polyclonal antibodies may be prepared by any known method or modifications of these methods including obtaining antibodies from patients. For example, a complex of an immunogen (an antigen against the protein) and a carrier protein is prepared and an animal is immunized by the complex according to the same manner as that described with respect to the above monoclonal antibody preparation. A material containing the antibody against is recovered from the immunized animal and the antibody is separated and purified.

As to the complex of the immunogen and the carrier protein to be used for immunization of an animal, any carrier protein and any mixing proportion of the carrier and a hapten can be employed as long as an antibody against the hapten, which is crosslinked on the carrier and used for immunization, is produced efficiently. For example, bovine serum albumin, bovine cycloglobulin, keyhole limpet hemocyanin, etc. may be coupled to an hapten in a weight ratio of about 0.1 part to about 20 parts, preferably, about 1 part to about 5 parts per 1 part of the hapten.

In addition, various condensing agents can be used for coupling of a hapten and a carrier. For example, glutaraldehyde, carbodiimide, maleimide activated ester, activated ester reagents containing thiol group or dithiopyridyl group, and the like find use with the present invention. The condensation product as such or together with a suitable carrier or diluent is administered to a site of an animal that permits the antibody production. For enhancing the antibody production capability, complete or incomplete Freund's adjuvant may be administered. Normally, the protein is administered once every 2 weeks to 6 weeks, in total, about 3 times to about 10 times.

The polyclonal antibody is recovered from blood, ascites and the like, of an animal immunized by the above method. The antibody titer in the antiserum can be measured according to the same manner as that described above with respect to the supernatant of the hybridoma culture. Separation and purification of the antibody can be carried out according to the same separation and purification method of immunoglobulin as that described with respect to the above monoclonal antibody.

The protein used herein as the immunogen is not limited to any particular type of immunogen. For example, a protein expressed resulting from a virus infection (further including a gene having a nucleotide sequence partly altered) can be used as the immunogen. Further, fragments of the protein may be used. Fragments may be obtained by any methods including, but not limited to expressing a fragment of the gene, enzymatic processing of the protein, chemical synthesis, and the like.

### XIII. Drug Screening

In some embodiments, the present invention provides drug screening assays (e.g., to screen for sST2 formulations). The screening methods of the present invention utilize gene expression maps identified using the methods of the present invention (e.g., including but not limited to, sST2). For example, in some embodiments, the present invention provides methods of screening for compound that alter (e.g., increase or decrease) the expression of virus-induced gene expression maps (profiles). In some embodiments, candidate compounds are antibodies that specifically bind to a protein encoded by a virus-induced gene of the present invention.

In one screening method, candidate compounds are evaluated for their ability to alter virus-induced gene expression by contacting a compound with a cell expressing a virus induced protein and then assaying for the effect of the candidate compounds on expression. In some embodiments, the effect of candidate compounds on expression of a virus induced gene is assayed for by detecting the level of mRNA expressed by the cell. mRNA expression can be detected by any suitable method. In other embodiments, the effect of candidate compounds on expression of virus induced genes is assayed by measuring the level of polypeptide encoded by the virus induced genes. The level of polypeptide expressed can be measured using any suitable method, including but not limited to, those disclosed herein.

Specifically, the present invention provides screening methods for identifying modulators, i.e., candidate or test compounds or agents (e.g., proteins, peptides, peptidomimetics, peptoids, small molecules or other drugs) which bind to virus induced gene products of the present invention, have an inhibitory (or stimulatory) effect on, for example, gene expression or gene product activity, or have a stimulatory or inhibitory effect on, for example, the expression or activity of a virus induced gene substrate. Compounds thus identified can be used to modulate the activity of target gene products (e.g., virus induced genes) either directly or indirectly in a therapeutic protocol, to elaborate the biological function of the target gene product, or to identify compounds that disrupt normal target gene interactions. Compounds which inhibit the activity or expression of virus induced genes are useful in the treatment of virus infections.

In one embodiment, the invention provides assays for screening candidate or test compounds that are substrates of a virus induced protein or polypeptide or a biologically active portion thereof. In another embodiment, the invention provides assays for screening candidate or test compounds that bind to or modulate the activity of a virus induced protein or polypeptide or a biologically active portion thereof.

The test compounds of the present invention can be obtained using any of the numerous approaches in combinatorial library methods, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J. Med. Chem. 37: 2678 85 (1994)); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (Lam (1997) Antivirus induced Drug Des. 12:145).

Numerous examples of methods for the synthesis of molecular libraries have been reported, for example in: DeWitt et al., Proc. Natl. Acad. Sci. U.S.A. 90:6909 (1993); Erb et al., Proc. Natl. Acad. Sci. USA 91:11422 (1994); Zuckermann et al., J. Med. Chem. 37:2678 (1994); Cho et al., Science 261:1303 (1993); Carrell et al., Angew. Chem. Int. Ed. Engl. 33.2059 (1994); Carell et al., Angew. Chem. Int. Ed. Engl. 33:2061 (1994); and Gallop et al., J. Med. Chem. 37:1233 (1994).

Libraries of compounds may be presented in solution (e.g., Houghten, Biotechniques 13:412 421 (1992)), or on beads (Lam, Nature 354:82 84 (1991)), chips (Fodor, Nature 364:555 556 (1993)), bacteria or spores (U.S. Pat. No. 5,223,409; herein incorporated by reference), plasmids (Cull et al., Proc. Natl. Acad. Sci. USA 89:18651869 (1992)) or on phage (Scott and Smith, Science 249:386 390 (1990); Devlin Science 249:404 406 (1990); Cwirla et al., Proc. Natl. Acad. Sci. 87:6378 6382 (1990); Felici, J. Mol. Biol. 222:301 (1991)).

In one embodiment, an assay is a cell-based assay in which a cell that expresses a virus induced protein or biologically active portion thereof is contacted with a test compound, and the ability of the test compound to the modulate virus induced protein activity is determined. Determining the ability of the test compound to modulate virus induced protein activity can be accomplished by monitoring, for example, changes in enzymatic activity. The cell, for example, can be of mammalian origin.

The ability of the test compound to modulate a virus induced protein binding to a compound, e.g., a virus induced substrate, can also be evaluated. This can be accomplished, for example, by coupling the compound, e.g., the substrate, with a radioisotope or enzymatic label such that binding of the compound, e.g., the substrate, can be determined by detecting the labeled compound, e.g., substrate, in a complex.

Alternatively, the virus induced protein is coupled with a radioisotope or enzymatic label to monitor the ability of a test compound to modulate virus induced protein binding to a substrate in a complex. For example, compounds (e.g., substrates) can be labeled with ¹²⁵I, ³⁵S, ¹⁴C, or ³H, either directly or indirectly, and the radioisotope detected by direct counting of radioemission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a compound to interact with a virus induced protein with or without the labeling of any of the interactants can be evaluated. For example, a microphysiometer can be used to detect the interaction of a compound with a virus induced marker without the labeling of either the compound or the virus induced marker (McConnell et al. Science 257:1906 1912 (1992)). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and markers.

In yet another embodiment, a cell-free assay is provided in which a virus induced marker protein or biologically active portion thereof is contacted with a test compound and the ability of the test compound to bind to the virus induced marker protein or biologically active portion thereof is evaluated. Preferred biologically active portions of the virus induced marker proteins to be used in assays of the present invention include fragments that participate in interactions with substrates or other proteins, e.g., fragments with high surface probability scores.

Cell-free assays involve preparing a reaction mixture of the target gene protein and the test compound under conditions and for a time sufficient to allow the two components to interact and bind, thus forming a complex that can be removed and/or detected.

The interaction between two molecules can also be detected, e.g., using fluorescence energy transfer (FRET) (see, for example, Lakowicz et al., U.S. Pat. No. 5,631,169; Stavrianopoulos et al., U.S. Pat. No. 4,968,103; each of which is herein incorporated by reference). A fluorophore label is selected such that a first donor molecule's emitted fluorescent energy will be absorbed by a fluorescent label on a second, ‘acceptor’ molecule, which in turn is able to fluoresce due to the absorbed energy.

Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FRET binding event can be conveniently measured through standard fluorometric detection means well known in the art (e.g., using a fluorimeter).

In another embodiment, determining the ability of the virus induced marker protein to bind to a target molecule can be accomplished using real-time Biomolecular Interaction Analysis (BIA) (see, e.g., Sjolander and Urbaniczky, Anal. Chem. 63:2338 2345 (1991) and Szabo et al. Curr. Opin. Struct. Biol. 5:699 705 (1995)). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal that can be used as an indication of real-time reactions between biological molecules.

In one embodiment, the target gene product or the test substance is anchored onto a solid phase. The target gene product/test compound complexes anchored on the solid phase can be detected at the end of the reaction. Preferably, the target gene product can be anchored onto a solid surface, and the test compound, (which is not anchored), can be labeled, either directly or indirectly, with detectable labels discussed herein.

It may be desirable to immobilize virus induced markers, an anti-virus induced marker antibody or its target molecule to facilitate separation of complexed from non-complexed forms of one or both of the proteins, as well as to accommodate automation of the assay. Binding of a test compound to a virus induced marker protein, or interaction of a virus induced marker protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase-virus induced marker fusion proteins or glutathione-S-transferase/target fusion proteins can be adsorbed onto glutathione Sepharose beads (Sigma Chemical, St. Louis, Mo.) or glutathione-derivatized microtiter plates, which are then combined with the test compound or the test compound and either the non-adsorbed target protein or virus induced marker protein, and the mixture incubated under conditions conducive for complex formation (e.g., at physiological conditions for salt and pH). Following incubation, the beads or microtiter plate wells are washed to remove any unbound components, the matrix immobilized in the case of beads, complex determined either directly or indirectly, for example, as described above.

Alternatively, the complexes can be dissociated from the matrix, and the level of virus induced markers binding or activity determined using standard techniques. Other techniques for immobilizing either virus induced marker proteins or a target molecule on matrices include using conjugation of biotin and streptavidin. Biotinylated virus induced marker protein or target molecules can be prepared from biotin-NHS(N-hydroxy-succinimide) using techniques known in the art (e.g., biotinylation kit, Pierce Chemicals, Rockford, EL), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-IgG antibody).

This assay is performed utilizing antibodies reactive with virus induced marker protein or target molecules but which do not interfere with binding of the virus induced markers protein to its target molecule. Such antibodies can be derivatized to the wells of the plate, and unbound target or virus induced markers protein trapped in the wells by antibody conjugation. Methods for detecting such complexes, in addition to those described above for the GST-immobilized complexes, include immunodetection of complexes using antibodies reactive with the virus induced marker protein or target molecule, as well as enzyme-linked assays which rely on detecting an enzymatic activity associated with the virus induced marker protein or target molecule.

Alternatively, cell free assays can be conducted in a liquid phase. In such an assay, the reaction products are separated from unreacted components, by any of a number of standard techniques, including, but not limited to: differential centrifugation (see, for example, Rivas and Minton, Trends Biochem Sci 18:284 7 (1993)); chromatography (gel filtration chromatography, ion-exchange chromatography); electrophoresis (see, e.g., Ausubel et al., eds. Current Protocols in Molecular Biology 1999, J. Wiley: New York.); and immunoprecipitation (see, for example, Ausubel et al., eds. Current Protocols in Molecular Biology 1999, J. Wiley: New York). Such resins and chromatographic techniques are known to one skilled in the art (See e.g., Heegaard J. Mol. Recognit 11: 141 8 (1998); Hageand Tweed J. Chromatogr. Biomed. Sci. Appl 699:499 525 (1997)). Further, fluorescence energy transfer may also be conveniently utilized, as described herein, to detect binding without further purification of the complex from solution.

The assay can include contacting the virus induced marker proteins or biologically active portion thereof with a known compound that binds the virus induced marker to form an assay mixture, contacting the assay mixture with a test compound, and determining the ability of the test compound to interact with a virus induced marker protein, wherein determining the ability of the test compound to interact with a virus induced marker protein includes determining the ability of the test compound to preferentially bind to virus induced markers or biologically active portion thereof, or to modulate the activity of a target molecule, as compared to the known compound.

To the extent that virus induced markers can, in vivo, interact with one or more cellular or extracellular macromolecules, such as proteins, inhibitors of such an interaction are useful. A homogeneous assay can be used can be used to identify inhibitors.

For example, a preformed complex of the target gene product and the interactive cellular or extracellular binding partner product is prepared such that either the target gene products or their binding partners are labeled, but the signal generated by the label is quenched due to complex formation (see, e.g., U.S. Pat. No. 4,109,496, herein incorporated by reference, that utilizes this approach for immunoassays). The addition of a test substance that competes with and displaces one of the species from the preformed complex will result in the generation of a signal above background. In this way, test substances that disrupt target gene product-binding partner interaction can be identified. Alternatively, virus induced markers protein can be used as a “bait protein” in a two-hybrid assay or three-hybrid assay (see, e.g., U.S. Pat. No. 5,283,317; Zervos et al., Cell 72:223 232 (1993); Madura et al., J. Biol. Chem. 268.12046 12054 (1993); Bartel et al., Biotechniques 14:920 924 (1993); Iwabuchi et al., Oncogene 8:1693 1696 (1993); and Brent WO 94/10300; each of which is herein incorporated by reference), to identify other proteins, that bind to or interact with virus induced markers (“virus induced marker-binding proteins” or “virus induced marker-bp”) and are involved in virus induced marker activity. Such virus induced marker-bps can be activators or inhibitors of signals by the virus induced marker proteins or targets as, for example, downstream elements of a virus induced markers-mediated signaling pathway.

Modulators of virus induced markers expression can also be identified. For example, a cell or cell free mixture is contacted with a candidate compound and the expression of virus induced marker mRNA or protein evaluated relative to the level of expression of virus induced marker mRNA or protein in the absence of the candidate compound. When expression of virus induced marker mRNA or protein is greater in the presence of the candidate compound than in its absence, the candidate compound is identified as a stimulator of virus induced marker mRNA or protein expression. Alternatively, when expression of virus induced marker mRNA or protein is less (i.e., statistically significantly less) in the presence of the candidate compound than in its absence, the candidate compound is identified as an inhibitor of virus induced marker mRNA or protein expression. The level of virus induced marker mRNA or protein expression can be determined by methods described herein for detecting virus induced marker mRNA or protein.

A modulating agent can be identified using a cell-based or a cell free assay, and the ability of the agent to modulate the activity of a virus induced marker protein can be confirmed in vivo, e.g., in an animal such as an animal model for a disease (e.g., an animal with dengue fever), or cells from a dengue fever virus induced cell line.

This invention further pertains to novel agents identified by the above-described screening assays. Accordingly, it is within the scope of this invention to further use an agent identified as described herein (e.g., a virus induced marker modulating agent, a virus induced marker specific antibody, or a virus induced marker-binding partner) in an appropriate animal model (such as those described herein) to determine the efficacy, toxicity, side effects, or mechanism of action, of treatment with such an agent. Furthermore, novel agents identified by the above-described screening assays can be, e.g., used for treatments as described herein.

### EXPERIMENTAL

### Example 1

### Patient Population of Clinical Trial

Thirty five Venezuelan patients with suspected dengue virus infection were included in this study. All patients were enrolled in a study protocol conducted by the University of Massachusetts Medical School (UMMS), Worcester, Mass., USA and Banco Municipal de Sangre del Distrito Capital (BMS), Caracas, Venezuela. Cardier et al., “Proinflammatory factors present in sera from patients with acute dengue infection induce activation and apoptosis of human microvascular endothelial cells: possible role of TNFalpha in endothelial cell damage in dengue” Cytokine 30:359-365 (2005). Written informed consent was obtained from all subjects. Criteria for enrollment included: presence of a febrile illness, with no evidence of other defined infections. Febrile patients attended the consult daily until 2 days after the fever resolved. A final consult was performed at least 2 weeks after the onset of symptoms. Blood samples for hematology, coagulation tests, serology and biochemical analysis were obtained daily. Serum and plasma samples were separated in aliquots and stored at −70° C. for analysis. Fourteen healthy donors from BMS and UMMS were used as controls for normal sST2 protein levels.

### Example 2

### Clinical Record and Laboratory Analysis

A complete clinical exam and routine laboratory tests were performed each day on the patients described in Example 1. Based on corporal temperature, we defined “fever day zero (0)” as the day of defervescence; days before defervescence were numbered as fever days −1, −2 and days after defervescence were numbered +1, +2. A thorax/abdomen ultrasound study was performed on day +1. Peripheral blood studies were performed using Gen-S autoanalyzer (Beckman-Coulter). Thrombin time (TT) was measured in plasma samples in a STA Compact automated coagulation analyzer (Diagnostica Stago) and compared against control TT obtained from healthy donors (ΔTT=TTpatient−TTcontrol). Fibrinogen levels were measured in plasma samples by Clauss method (Diagnostica Stago). Aspartate aminotransferase (AST) and alanine transaminase (ALT) were measured in serum samples using Sigma-transaminase kit (Sigma-Aldrich).

### Example 3

### Dengue Fever Diagnosis

Dengue RNA was isolated from febrile serum samples using the QIAmp Viral RNA kit (QIAGEN). Dengue virus serotype specific revere transcription and polymerase chain reaction (RT-PCR) was performed using the One-step PCR kit (QIAGEN) and primers adapted to a one-step RT-PCR using reverse primer and serotype specific forward primers. Lanciotti et al., “Rapid detection and typing of dengue viruses from clinical samples by using reverse transcriptase polymerase chain reaction” J Clin Microbiol 30:545-551 (1992). Dengue antibodies were measured by ELISA (IgM) and hemagglutination inhibition assay (HI), at the Instituto Nacional de Higiene Rafael Rangel (National Reference Laboratory), Caracas, Venezuela.

Patients were classified as Dengue or as an Other Febrile Illness (OFI) based on the detection of genomic dengue RNA, presence of IgM antibodies and/or a ≧four-fold increase in HI levels in the final sample (S2) compared to the first sample (S1). The HI levels were used to further classify dengue patients as a primary infection (HI titer≦1:1280) or secondary infection (HI titer>1:1280). “Joint WHO HQ/SEAROP/WPRO meeting on DengueNet implementation in South-East Asiand the Western Pacific, Kuala Lumpur, 11-13 Dec. 2003” Wkly Epidemiol Rec 78:346-347 (2003).

### Example 4

### Quantification of Soluble ST2 Protein

Serum levels of sST2 were measured by ELISA (MBL Int.) following the manufacturer's instructions. Serum samples during febrile (fever days −2 and −1), defervescence (fever day 0), post-febrile (fever days +1 and +2) and convalescence (at least 2 weeks after the onset) stages were tested for soluble ST2 protein levels. For each healthy donor a single serum sample was analyzed, to generate basal levels of sST2.

The Mann-Whitney U or Kruskal-Wallis tests were used for comparisons between groups for continuous variables not normally distributed. X2 was used to compare categorical data. Spearman's correlation was used to examine correlations between continuous variables. SPSS 14.0 for Windows (Copyright SPSS Inc. 1989-2005) software for the statistical analysis.

### Example 5

### Clinical Characteristics of Dengue Fever Infected Patients

The characteristics of the patients enrolled in a dengue fever study protocol are shown in Table 1.

TABLE 1
Patient Clinical Profiles

| | | Dengue | | |
|---|---|---|---|---|
| | OFI | All | Primary | Secondary |
| Classification ^{a} | (N = 11) | (N = 24) | (N = 10) | (N = 13) |
| Age ^{b} | 33 | 22 | 22 | 25 |
| | (13-56) | (9-55) | (9-33) | (11-55) |
| Sex (F:M) | (8:3) | (8:16) | (5:5) | (2:11) |
| Clinical Sign and Symptoms ^{c} | | | | |
| Petechiae ^{d} | 0 | 18 | 7 | 10 |
| Hemorrhage ^{e} | 3 | 6 | 2 | 4 |
| Vascular Leakage | 0 | 2 | 0 | 2 |
| (ultrasound) | | | | |
| Edema | 0 | 7 | 3 | 3 |
| Laboratory Parameters ^{f} | | | | |
| Minimum platelet count | 187 ± 18 | 90 ± 10 | 118 ± 11 | 69 ± 14 |
| (×10³/μl) | (147-228) | (69-111) | (92-144) | (39-98) |
| Minimum WBC | 4.4 ± 0.7 | 2.6 ± 0.2 | 2.6 ± 0.3 | 2.7 ± 0.3 |
| (×10³/μl) | (3.0-5.9) | (2.1-3.1) | (1.8-3.3) | (2.0-3.5) |
| Maximum AST | 69.4 ± 37.6 | 186.5 ± 33.5 | 93.4 ± 10.6 | 249.0 ± 54.1 |
| (U/ml) | (14.3-153.1) | (117.2-255.8) | (69.4-117.3) | (131.1-367.0) |
| Maximum ALT | 61.2 ± 29.7 | 137.9 ± 25.0 | 70.1 ± 8.2 | 189.0 ± 40.8 |
| (U/ml) | (4.9-127.3) | (86.1-189.7) | (51.6-88.6) | (100.1-277.9) |
| Maximum thrombin time | 1.1 ± 0.5 | 12.0 ± 2.9 | 5.2 ± 1.0 | 17.2 ± 4.8 |
| difference (ΔTT, s) | (0.1-2.3) | (6.0-17.9) | (2.8-7.5) | (6.7-29.6) |
| Minimum fibrinogen | 387 ± 27 | 283 ± 12 | 299 ± 20 | 277 ± 16 |
| (mg/dL) | (327-446) | (257-309) | (255-344) | (243-312) |

^{a} Patients were classified according to dengue viral RNA and IgM detection. Dengue patients were positive for both dengue viral RNA and IgM; OFI patients were negative for both parameters. Primary patients had HI titers ≦1:1280 and secondary patients had HI titers >1280. One dengue patient could not be classified as primary or secondary.
^{b} Age in years (median and range).
^{c} Frequency of patients with each sign/symptoms during the period of study.
^{d} Petechiae: positive tourniquet test and/or spontaneous petechiae.
^{e} Types of Hemorrhages: OFI (epistaxis, hematoma); primary (gum bleeding); secondary (epistaxis, gum bleeding, hematoma, hematuria).
^{f} Average value ± standard deviation and 95% confidence interval.

Patients were classified based on detection of dengue virus specific IgM and genomic dengue RNA in serum. Patients positive for IgM or genomic RNA were classified as “dengue” and patients that did not meet these criteria were classified as “other febrile illness (OFI)”. Dengue patients were further subclassified as primary or secondary infections based on hemaglutination inhibition assay (HI) titer. The present study included a group of eleven OFI and twenty four dengue patients; ten dengue patients had primary infections and thirteen had secondary infections and one was not sub-classified. All dengue patients were classified as DF according to the World Health Organization case definition. “Joint WHO HQ/SEAROP/WPRO meeting on DengueNet implementation in South-East Asiand the Western Pacific, Kuala Lumpur, 11-13 Dec. 2003” Wkly Epidemiol Rec 78:346-347 (2003).

The frequency of petechiae (p<0.001), edema (p=0.045) and rash (p=0.002) were higher in dengue patients compared with OFI patients. Significant differences in the minimum white blood cell (WBC) count (p=0.011), minimum platelets count (p<0.001), maximum aspartate aminotransferase (AST) (p<0.001), maximum alanine transaminase (ALT) (p=0.004), maximum prolonged thrombin time difference (ΔTT) (p<0.001) and minimum fibrinogen levels (p<0.001) were observed between OFI and dengue patients. Further, when primary and secondary infected patients were compared, we found significant differences in the minimum platelets count (p=0.018), maximum AST (p=0.042) and maximum ALT (p=0.049).

### Example 6

### Detection of sST2 in Dengue Fever Patients

The patients described in Examples 1 & 2 were assayed for serum sST2 protein. In summary, the sST2 levels (pg/ml) in serum of healthy donors and/or OFI patients were lower than sST2 levels in patients experiencing an acute stage of a dengue fever infection. See, FIG. 4.

The sST2 levels were elevated during late febrile days of the disease, reaching maximum values on fever days −1 and 0, followed by decrease in sST2 protein levels close to healthy donors values by the convalescent day (at least 15 days after onset of the disease) to levels similar to those of healthy donors. The increase in sST2 protein levels were statistically significant for the all dengue patients (p<0.001) but not for the OFI group, indicating a specific increase in sST2 protein levels during acute stage of dengue virus infections. sST2 protein levels were significantly higher on fever days −1 and 0 (p<0.001) as compared to convalescence in all dengue patients while for OFI there were no statistically significant differences in sST2 levels between stages. We also found statistically higher sST2 levels in all dengue patients compared to OFI at fever days −1 (p=0.0088) and 0 (p=0.0004) suggesting that sST2 protein levels are preferentially increased in dengue virus infections.

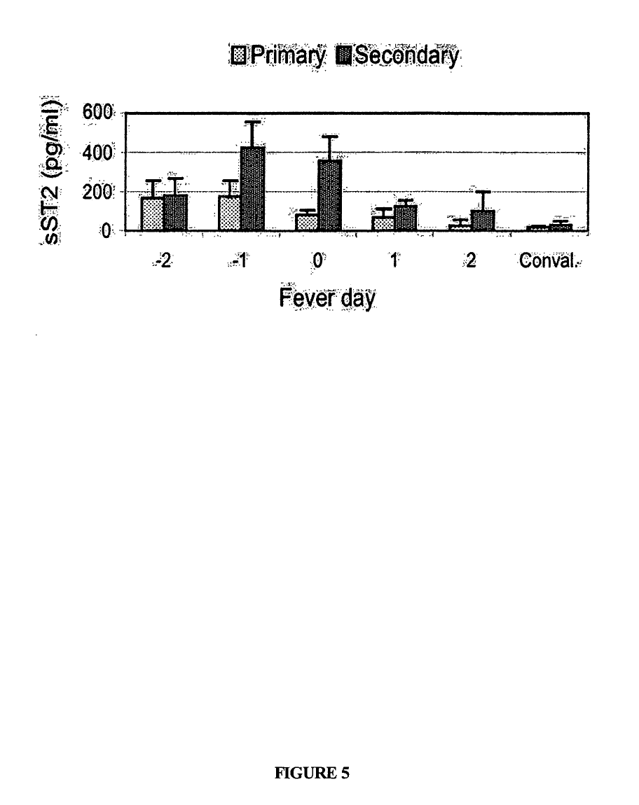

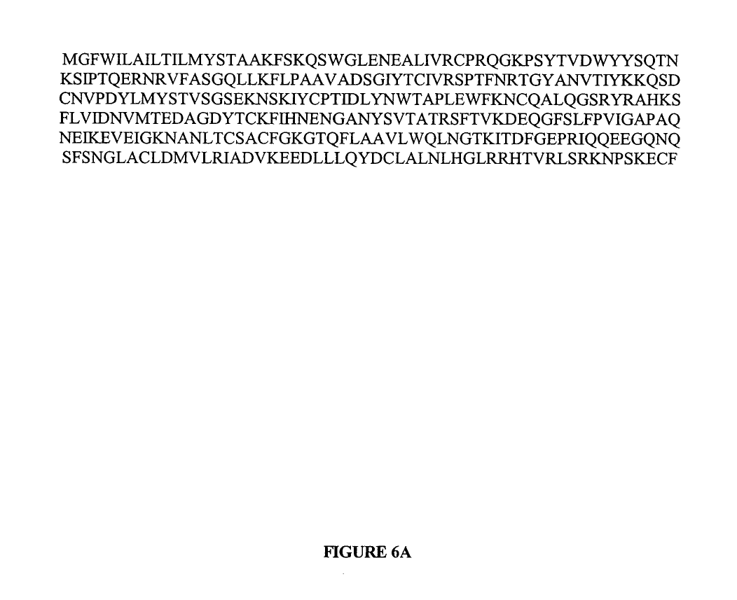

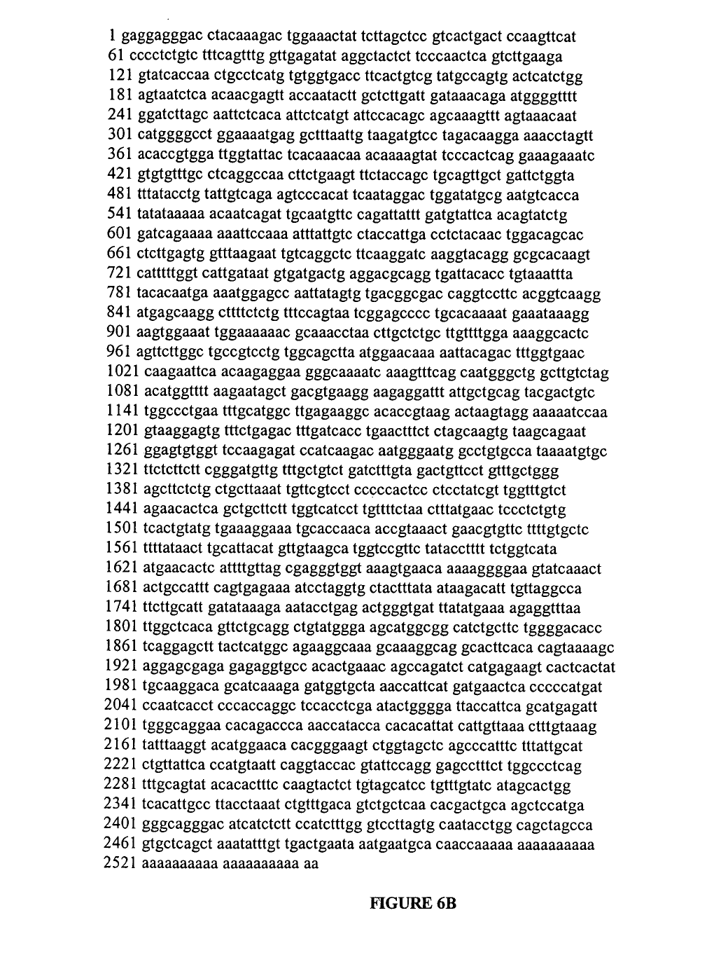

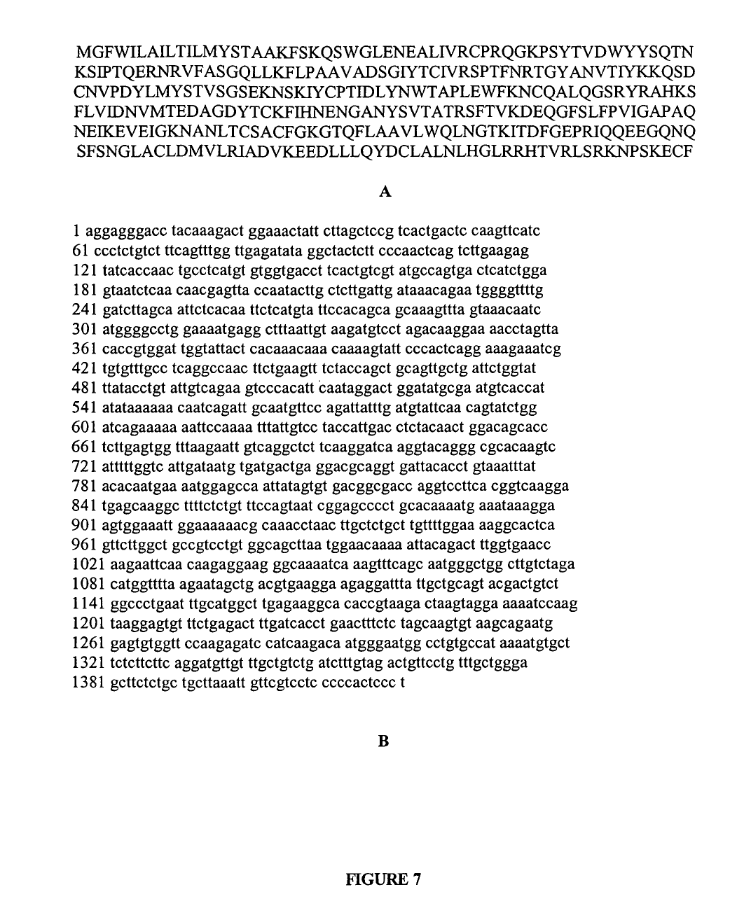

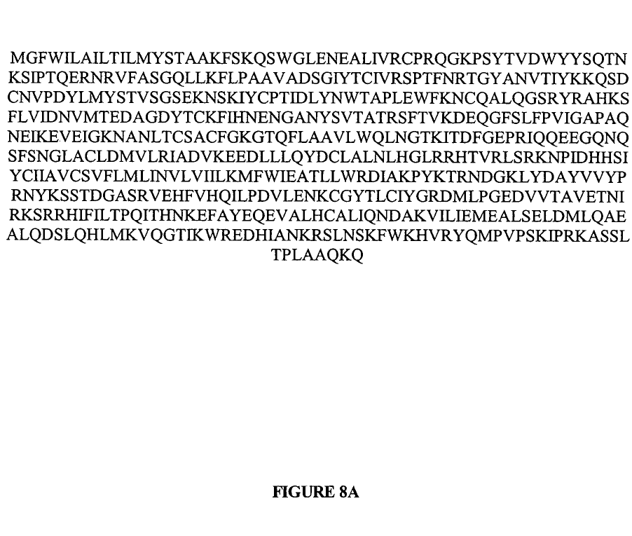

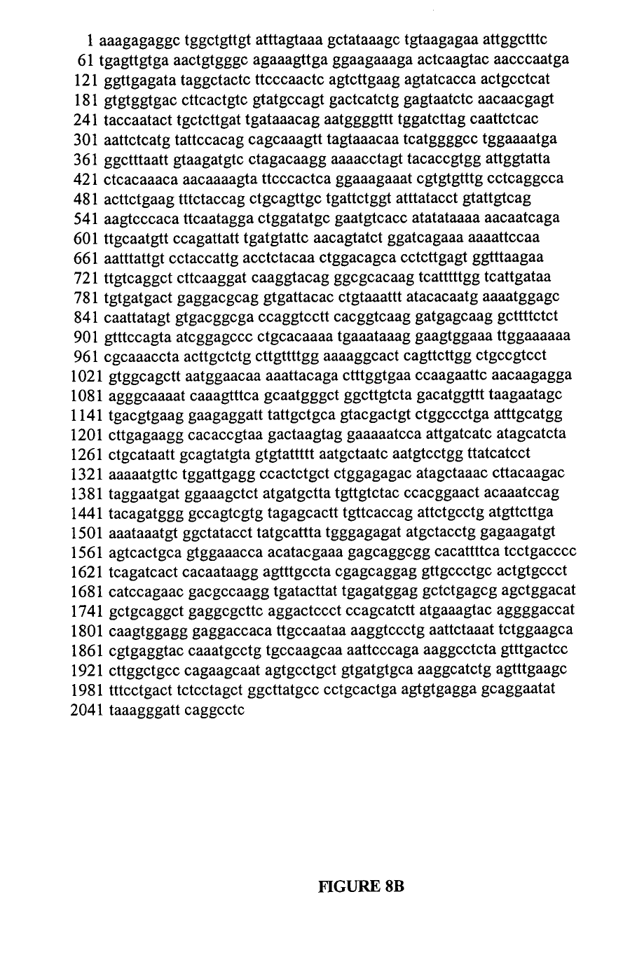

Analysis of dengue fever patients sub-classified as either primary or secondary infections, found a statistically significant higher sST2 levels in secondary infections on fever days −1 (p<0.001) and 0 (p<0.01) as compared to the sST2 levels on convalescence, a result that was not observed in primary infections. Higher sST2 levels in secondary infections as compared to primary infections were also found at fever days −1 (p=0.047) and 0 (p=0.030). See, FIG. 5.

### Example 7

### Correlations Between sST2 and Laboratory Parameters

The data collected in accordance with Examples 2 & 3 were subjected to correlation analysis. Specifically, sST2 protein levels were correlated with numerous laboratory parameters known to be associated with disease severity in dengue virus infections. Correlations were assessed between the sST2 value (pg/ml) and the corresponding value for each laboratory parameter for the same day of the disease.

In all dengue virus infected patients a negative correlation was found between sST2 protein levels and WBC (r=−0.357; p<0.01) and platelet counts (r=−0.504; p<0.01). In contrast, a positive correlation was found between sST2 protein levels and ΔTT (r=0.366; p<0.01), AST (r=0.462; p<0.01) and ALT (r=0.237; p<0.05).

In the secondary infected patients a negative correlation was found between sST2 protein levels and WBC (r=−0.505; p<0.01) and platelet counts (r=−0.553; p<0.01). In contrast, a positive correlation was found between sST2 and AST (r=0.496; p<0.01) and ΔTT (r=0.306; p<0.05).

In the primary infected patients, only a positive correlation was found between sST2 and AST (r=0.312; p<0.05) and ΔTT (r=0.356; p<0.05).

We claim:

1. A biomarker for a dengue fever infection, wherein said biomarker comprises a protein level elevated to at least 1.5 times that of other febrile illnesses.

2. The biomarker of claim 1, wherein said protein comprises a soluble interleukin 1 receptor-like 1 protein.

3. The biomarker of claim 1, wherein said elevated protein level is detected in a biological sample.

4. The biomarker of claim 3, wherein said biological sample is selected from the group consisting of whole blood, plasma, serum, and a tissue biopsy.

5. The biomarker of claim 4, wherein said tissue biopsy comprises a somatic cell.

6. A biomarker for a dengue fever infection, wherein said biomarker comprises a nucleic acid level elevated to at least 1.5 times that of other febrile illnesses.

7. The biomarker of claim 6, wherein said nucleic acid encodes a soluble interleukin 1 receptor-like 1 protein.

8. The biomarker of claim 6, wherein said elevated nucleic acid level is detected in a biological sample.

9. The biomarker of claim 8, wherein said biological sample is selected from the group consisting of whole blood, plasma, serum, and a tissue biopsy.

10. The biomarker of claim 9, wherein said tissue biopsy comprises a somatic cell.

11. A method, comprising:
a) providing;
i) a patient suspected of having a dengue fever infection;
ii) a biological sample derived from said patient, wherein said sample is capable of comprising a soluble interleukin 1 receptor-like 1 protein; and
b) detecting said protein in said sample.

12. The method of claim 11, wherein said detecting identifies that said protein is elevated to at least 1.5 times relative to other febrile illnesses.

13. The method of claim 11, wherein said detecting comprises an antibody, wherein said antibody is directed to said protein.

14. The method of claim 13, wherein said antibody is labeled.

15. The method of claim 11, wherein said detecting comprises a primer, wherein said primer is complementary to a nucleic acid encoding said protein.

16. The method of claim 12, wherein identification of said elevated protein diagnoses said dengue fever infection.

17. A kit, comprising:
a) a reagent capable of detecting a soluble interleukin 1 receptor-like 1 protein; and,
b) a sheet of instructions capable of diagnosing a virus infection based upon said detected protein.

18. The kit of claim 17, wherein said instructions are capable of diagnosing a primary dengue fever infection.

19. The kit of claim 17, wherein said instructions are capable of diagnosing a secondary dengue fever infection.

20. The kit of claim 17, wherein said reagent comprises an antibody, wherein said antibody is directed to said protein.

21. A kit, comprising:
a) a reagent capable of detecting a soluble interleukin 1 receptor-like 1 nucleic acid; and,
b) a sheet of instruction capable of diagnosing a virus infection based upon said detected nucleic acid.

22. The kit of claim 21, wherein said instructions are capable of diagnosing a primary dengue fever infection.

23. The kit of claim 21, wherein said instructions are capable of diagnosing a secondary dengue fever infection.

24. The kit of claim 21, wherein said reagent comprises a primer, wherein said primer is complementary to said nucleic acid.


SEQUENCE LISTING (7 sequences)

SEQ ID NO: 1 - protein, 328 aa, Homo sapiens
MGFWILAILTILMYSTAAKFSKQSWGLENEALIVRCPRQGKPSYTVDWYYSQTNKSIPTQERNRVFASGQLLKFLPAAVADSGIYTCIVRSPTFNRTGYANVTIYKKQSDCNVPDYLMYSTVSGSEKNSKIYCPTIDLYNWTAPLEWFKNCQALQGSRYRAHKSFLVIDNVMTEDAGDYTCKFIHNENGANYSVTATRSFTVKDEQGFSLFPVIGAPAQNEIKEVEIGKNANLTCSACFGKGTQFLAAVLWQLNGTKITDFGEPRIQQEEGQNQSFSNGLACLDMVLRIADVKEEDLLLQYDCLALNLHGLRRHTVRLSRKNPSKECF

SEQ ID NO: 2 - DNA, 2542 bp, Homo sapiens
gaggagggacctacaaagactggaaactattcttagctccgtcactgactccaagttcatcccctctgtctttcagtttggttgagatataggctactcttcccaactcagtcttgaagagtatcaccaactgcctcatgtgtggtgaccttcactgtcgtatgccagtgactcatctggagtaatctcaacaacgagttaccaatacttgctcttgattgataaacagaatggggttttggatcttagcaattctcacaattctcatgtattccacagcagcaaagtttagtaaacaatcatggggcctggaaaatgaggctttaattgtaagatgtcctagacaaggaaaacctagttacaccgtggattggtattactcacaaacaaacaaaagtattcccactcaggaaagaaatcgtgtgtttgcctcaggccaacttctgaagtttctaccagctgcagttgctgattctggtatttatacctgtattgtcagaagtcccacattcaataggactggatatgcgaatgtcaccatatataaaaaacaatcagattgcaatgttccagattatttgatgtattcaacagtatctggatcagaaaaaaattccaaaatttattgtcctaccattgacctctacaactggacagcacctcttgagtggtttaagaattgtcaggctcttcaaggatcaaggtacagggcgcacaagtcatttttggtcattgataatgtgatgactgaggacgcaggtgattacacctgtaaatttatacacaatgaaaatggagccaattatagtgtgacggcgaccaggtccttcacggtcaaggatgagcaaggcttttctctgtttccagtaatcggagcccctgcacaaaatgaaataaaggaagtggaaattggaaaaaacgcaaacctaacttgctctgcttgttttggaaaaggcactcagttcttggctgccgtcctgtggcagcttaatggaacaaaaattacagactttggtgaaccaagaattcaacaagaggaagggcaaaatcaaagtttcagcaatgggctggcttgtctagacatggttttaagaatagctgacgtgaaggaagaggatttattgctgcagtacgactgtctggccctgaatttgcatggcttgagaaggcacaccgtaagactaagtaggaaaaatccaagtaaggagtgtttctgagactttgatcacctgaactttctctagcaagtgtaagcagaatggagtgtggttccaagagatccatcaagacaatgggaatggcctgtgccataaaatgtgcttctcttcttcgggatgttgtttgctgtctgatctttgtagactgttcctgtttgctgggagcttctctgctgcttaaattgttcgtcctcccccactccctcctatcgttggtttgtctagaacactcagctgcttctttggtcatccttgttttctaactttatgaactccctctgtgtcactgtatgtgaaaggaaatgcaccaacaaccgtaaactgaacgtgttcttttgtgctcttttataacttgcattacatgttgtaagcatggtccgttctatacctttttctggtcataatgaacactcattttgttagcgagggtggtaaagtgaacaaaaaggggaagtatcaaactactgccatttcagtgagaaaatcctaggtgctactttataataagacatttgttaggccattcttgcattgatataaagaaatacctgagactgggtgatttatatgaaaagaggtttaattggctcacagttctgcaggctgtatgggaagcatggcggcatctgcttctggggacacctcaggagctttactcatggcagaaggcaaagcaaaggcaggcacttcacacagtaaaagcaggagcgagagagaggtgccacactgaaacagccagatctcatgagaagtcactcactattgcaaggacagcatcaaagagatggtgctaaaccattcatgatgaactcacccccatgatccaatcacctcccaccaggctccacctcgaatactggggattaccattcagcatgagatttgggcaggaacacagacccaaaccataccacacacattatcattgttaaactttgtaaagtatttaaggtacatggaacacacgggaagtctggtagctcagcccatttctttattgcatctgttattcaccatgtaattcaggtaccacgtattccagggagcctttcttggccctcagtttgcagtatacacactttccaagtactcttgtagcatcctgtttgtatcatagcactggtcacattgccttacctaaatctgtttgacagtctgctcaacacgactgcaagctccatgagggcagggacatcatctcttccatctttgggtccttagtgcaatacctggcagctagccagtgctcagctaaatatttgttgactgaataaatgaatgcacaaccaaaaaaaaaaaaaaaaaaaaaaaaaaaaaaaaaaaaa

SEQ ID NO: 3 - protein, 328 aa, Homo sapiens; identical to SEQ ID NO: 1

SEQ ID NO: 4 - DNA, 1421 bp, Homo sapiens
aggagggacctacaaagactggaaactattcttagctccgtcactgactccaagttcatcccctctgtctttcagtttggttgagatataggctactcttcccaactcagtcttgaagagtatcaccaactgcctcatgtgtggtgaccttcactgtcgtatgccagtgactcatctggagtaatctcaacaacgagttaccaatacttgctcttgattgataaacagaatggggttttggatcttagcaattctcacaattctcatgtattccacagcagcaaagtttagtaaacaatcatggggcctggaaaatgaggctttaattgtaagatgtcctagacaaggaaaacctagttacaccgtggattggtattactcacaaacaaacaaaagtattcccactcaggaaagaaatcgtgtgtttgcctcaggccaacttctgaagtttctaccagctgcagttgctgattctggtatttatacctgtattgtcagaagtcccacattcaataggactggatatgcgaatgtcaccatatataaaaaacaatcagattgcaatgttccagattatttgatgtattcaacagtatctggatcagaaaaaaattccaaaatttattgtcctaccattgacctctacaactggacagcacctcttgagtggtttaagaattgtcaggctcttcaaggatcaaggtacagggcgcacaagtcatttttggtcattgataatgtgatgactgaggacgcaggtgattacacctgtaaatttatacacaatgaaaatggagccaattatagtgtgacggcgaccaggtccttcacggtcaaggatgagcaaggcttttctctgtttccagtaatcggagcccctgcacaaaatgaaataaaggaagtggaaattggaaaaaacgcaaacctaacttgctctgcttgttttggaaaaggcactcagttcttggctgccgtcctgtggcagcttaatggaacaaaaattacagactttggtgaaccaagaattcaacaagaggaagggcaaaatcaaagtttcagcaatgggctggcttgtctagacatggttttaagaatagctgacgtgaaggaagaggatttattgctgcagtacgactgtctggccctgaatttgcatggcttgagaaggcacaccgtaagactaagtaggaaaaatccaagtaaggagtgtttctgagactttgatcacctgaactttctctagcaagtgtaagcagaatggagtgtggttccaagagatccatcaagacaatgggaatggcctgtgccataaaatgtgcttctcttcttcaggatgttgtttgctgtctgatctttgtagactgttcctgtttgctgggagcttctctgctgcttaaattgttcgtcctcccccactccct

SEQ ID NO: 5 - protein, 556 aa, Homo sapiens
MGFWILAILTILMYSTAAKFSKQSWGLENEALIVRCPRQGKPSYTVDWYYSQTNKSIPTQERNRVFASGQLLKFLPAAVADSGIYTCIVRSPTFNRTGYANVTIYKKQSDCNVPDYLMYSTVSGSEKNSKIYCPTIDLYNWTAPLEWFKNCQALQGSRYRAHKSFLVIDNVMTEDAGDYTCKFIHNENGANYSVTATRSFTVKDEQGFSLFPVIGAPAQNEIKEVEIGKNANLTCSACFGKGTQFLAAVLWQLNGTKITDFGEPRIQQEEGQNQSFSNGLACLDMVLRIADVKEEDLLLQYDCLALNLHGLRRHTVRLSRKNPIDHHSIYCIIAVCSVFLMLINVLVIILKMFWIEATLLWRDIAKPYKTRNDGKLYDAYVVYPRNYKSSTDGASRVEHFVHQILPDVLENKCGYTLCIYGRDMLPGEDVVTAVETNIRKSRRHIFILTPQITHNKEFAYEQEVALHCALIQNDAKVILIEMEALSELDMLQAEALQDSLQHLMKVQGTIKWREDHIANKRSLNSKFWKHVRYQMPVPSKIPRKASSLTPLAAQKQ

SEQ ID NO: 6 - DNA, 2058 bp, Homo sapiens
aaagagaggctggctgttgtatttagtaaagctataaagctgtaagagaaattggctttctgagttgtgaaactgtgggcagaaagttgaggaagaaagaactcaagtacaacccaatgaggttgagatataggctactcttcccaactcagtcttgaagagtatcaccaactgcctcatgtgtggtgaccttcactgtcgtatgccagtgactcatctggagtaatctcaacaacgagttaccaatacttgctcttgattgataaacagaatggggttttggatcttagcaattctcacaattctcatgtattccacagcagcaaagtttagtaaacaatcatggggcctggaaaatgaggctttaattgtaagatgtcctagacaaggaaaacctagttacaccgtggattggtattactcacaaacaaacaaaagtattcccactcaggaaagaaatcgtgtgtttgcctcaggccaacttctgaagtttctaccagctgcagttgctgattctggtatttatacctgtattgtcagaagtcccacattcaataggactggatatgcgaatgtcaccatatataaaaaacaatcagattgcaatgttccagattatttgatgtattcaacagtatctggatcagaaaaaaattccaaaatttattgtcctaccattgacctctacaactggacagcacctcttgagtggtttaagaattgtcaggctcttcaaggatcaaggtacagggcgcacaagtcatttttggtcattgataatgtgatgactgaggacgcaggtgattacacctgtaaatttatacacaatgaaaatggagccaattatagtgtgacggcgaccaggtccttcacggtcaaggatgagcaaggcttttctctgtttccagtaatcggagcccctgcacaaaatgaaataaaggaagtggaaattggaaaaaacgcaaacctaacttgctctgcttgttttggaaaaggcactcagttcttggctgccgtcctgtggcagcttaatggaacaaaaattacagactttggtgaaccaagaattcaacaagaggaagggcaaaatcaaagtttcagcaatgggctggcttgtctagacatggttttaagaatagctgacgtgaaggaagaggatttattgctgcagtacgactgtctggccctgaatttgcatggcttgagaaggcacaccgtaagactaagtaggaaaaatccaattgatcatcatagcatctactgcataattgcagtatgtagtgtatttttaatgctaatcaatgtcctggttatcatcctaaaaatgttctggattgaggccactctgctctggagagacatagctaaaccttacaagactaggaatgatggaaagctctatgatgcttatgttgtctacccacggaactacaaatccagtacagatggggccagtcgtgtagagcactttgttcaccagattctgcctgatgttcttgaaaataaatgtggctataccttatgcatttatgggagagatatgctacctggagaagatgtagtcactgcagtggaaaccaacatacgaaagagcaggcggcacattttcatcctgacccctcagatcactcacaataaggagtttgcctacgagcaggaggttgccctgcactgtgccctcatccagaacgacgccaaggtgatacttattgagatggaggctctgagcgagctggacatgctgcaggctgaggcgcttcaggactccctccagcatcttatgaaagtacaggggaccatcaagtggagggaggaccacattgccaataaaaggtccctgaattctaaattctggaagcacgtgaggtaccaaatgcctgtgccaagcaaaattcccagaaaggcctctagtttgactcccttggctgcccagaagcaatagtgcctgctgtgatgtgcaaaggcatctgagtttgaagctttcctgacttctcctagctggcttatgcccctgcactgaagtgtgaggagcaggaatattaaagggattcaggcctc

SEQ ID NO: 7 - protein, 8 aa, Artificial Sequence
  Synthetic
DYKDDDDK